(19) United States
(12) Patent Application Publication
(10) Pub. No.: US 2024/0138741 A1
(43) Pub. Date: May 2, 2024

(54) PACING ARTIFACT MITIGATION

(71) Applicant: Medtronic, Inc., Minneapolis, MN (US)

(72) Inventors: David J. Peichel, Minneapolis, MN (US); Timothy C. Aarons, Maple Grove, MN (US); Subham Ghosh, Blaine, MN (US); Michael T. Hemming, Kiowa, CO (US)

(21) Appl. No.: 18/370,166

(22) Filed: Sep. 19, 2023

Related U.S. Application Data
(60) Provisional application No. 63/420,273, filed on Oct. 28, 2022.

(51) Int. Cl. A61B 5/333

(52) U.S. Cl. CPC A61B 5/333; A61B 5/725

(57) ABSTRACT

Various embodiments of a system are disclosed. The system includes a sensing apparatus configured to monitor cardiac electrical activity of a patient and a computing apparatus operatively coupled to the sensing apparatus and configured to monitor cardiac electrical activity using the sensing apparatus to generate a cardiac signal over time, detect a pacing artifact in the cardiac signal, and determine to account for the pacing artifact when using the cardiac signal based on at least one pacing artifact characteristic of the pacing artifact in the cardiac signal. The computing apparatus is further configured to account for the pacing artifact when using the cardiac signal if it is determined to account for the pacing artifact.

### RELATED APPLICATIONS

This application claims the benefit of U.S. Provisional Application Ser. No. 63/420,273, filed Oct. 28, 2022, the entire contents of each of which are incorporated herein by reference.

### TECHNICAL FIELD

This disclosure generally relates to systems and method for pacing artifact mitigation in systems using external electrodes to monitor cardiac electrical activity of a patient.

### BACKGROUND

Implantable medical devices (IMDs), such as implantable pacemakers, cardioverters, defibrillators, pacemaker-cardioverter-defibrillators, or implantable cardiac monitors, provide therapeutic electrical stimulation to the heart and/or monitor electrical activity of the heart. IMDs may provide pacing to address bradycardia, or pacing or shocks in order to terminate tachyarrhythmia, such as tachycardia or fibrillation. In some cases, the medical device may sense intrinsic depolarizations of the heart, detect arrhythmia based on the intrinsic depolarizations (or absence thereof), and control delivery of electrical stimulation to the heart if arrhythmia is detected based on the intrinsic depolarizations.

IMDs may also provide cardiac resynchronization therapy (CRT), which is a form of pacing. CRT involves the delivery of pacing to the left ventricle, or both the left and right ventricles. The timing and location of the delivery of pacing pulses to the ventricle(s) may be selected to improve the coordination and efficiency of ventricular contraction.

Systems for implanting medical devices may include workstations or other equipment in addition to the implantable medical device itself. In some cases, these other pieces of equipment assist the physician or other technician with placing the intracardiac leads at particular locations on the heart. In some cases, the equipment provides information to the physician about the electrical activity of the heart and the location of the intracardiac lead. The equipment may perform similar functions as the medical device, including delivering electrical stimulation to the heart and sensing the depolarizations of the heart. In some cases, the equipment may include equipment for obtaining an electrocardiogram (ECG) via electrodes on the surface, or skin, of the patient. More specifically, the patient may have a plurality of electrodes on an ECG belt or vest that surrounds the torso of the patient. After the belt or vest has been secured to the torso, a physician can perform a series of tests to evaluate a patient's cardiac response. The evaluation process can include detection of a baseline rhythm in which no electrical stimuli is delivered to cardiac tissue and another rhythm after electrical stimuli is delivered to the cardiac tissue.

The ECG electrodes placed on the body surface of the patient may be used for various therapeutic purposes (e.g., cardiac resynchronization therapy) including optimizing lead location, pacing parameters, etc. based on one or more metrics derived from the signals captured by the ECG electrodes. For example, electrical heterogeneity information may be derived from electrical activation times computed from multiple electrodes on the body surface.

### SUMMARY

The techniques of this disclosure generally relate to systems and methods for mitigating artifacts in a cardiac signal of a patient. An exemplary system can include a sensing apparatus and a computing apparatus coupled to the sensing apparatus. The computing apparatus can be configured to detect a pacing artifact in a cardiac signal, determine whether to account for such pacing artifact, and account for the pacing artifact when using the cardiac signal if it is determined to account for the pacing artifact. In one or more embodiments, the computing apparatus can further be configured to account for the pacing artifact by removing the pacing artifact from the cardiac signal and replacing the removed pacing artifact in the cardiac signal with replacement data points in a desired arrangement such as provided by fitting the data points to a desired function.

In one example, aspects of this disclosure relate to a system that includes a sensing apparatus configured to monitor cardiac electrical activity of a patient; and a computing apparatus that includes one or more processors and operatively coupled to the sensing apparatus. The computing apparatus is configured to monitor cardiac electrical activity using the sensing apparatus to generate a cardiac signal over time, detect a pacing artifact in the cardiac signal, and determine to account for the pacing artifact when using the cardiac signal based on at least one pacing artifact characteristic of the pacing artifact in the cardiac signal. The computing apparatus can be further configured to account for the pacing artifact when using the cardiac signal if it is determined to account for the pacing artifact. Accounting for the pacing artifact includes removing the pacing artifact from the cardiac signal between a first data point at time T1 and a second data point at time T2 of the cardiac signal, and replacing the removed pacing artifact in the cardiac signal with replacement data points disposed along a straight line that extends between the first data point and the second data point of the cardiac signal to provide an optimized cardiac signal.

In another example, aspects of this disclosure relate to an implantable medical device that includes a housing, a computing apparatus disposed within the housing and including one or more processors, where the computing apparatus is operatively coupleable to a sensing apparatus configured to monitor cardiac electrical activity of a patient. The computing apparatus is further configured to monitor cardiac electrical activity using the sensing apparatus to generate a cardiac signal over time, detect a pacing artifact in the cardiac signal, determine to account for the pacing artifact when using the cardiac signal based on at least one pacing artifact characteristic of the pacing artifact in the cardiac signal, and account for the pacing artifact when using the cardiac signal if it is determined to account for the pacing artifact. Accounting for the pacing artifact includes removing the pacing artifact from the cardiac signal between a first data point at time T1 and a second data point at time T2 of the cardiac signal, and replacing the removed pacing artifact in the cardiac signal with replacement data points disposed along a straight line that extends between the first data point and the second data point of the cardiac signal to provide an optimized cardiac signal.

In another example, aspects of this disclosure relate to a method that includes monitoring cardiac electrical activity using a sensing apparatus to generate a cardiac signal over time, detecting a pacing artifact in the cardiac signal, and determining to account for the pacing artifact when using the cardiac signal based on at least one pacing artifact characteristic of the pacing artifact in the cardiac signal. The method further includes accounting for the pacing artifact when using the cardiac signal if it is determined to account for the pacing artifact. Accounting for the pacing artifact includes removing the pacing artifact from the cardiac signal between a first data point at time T1 and a second data point at time T2 of the cardiac signal, and replacing the removed pacing artifact in the cardiac signal with replacement data points disposed along a straight line that extends between the first data point and the second data point of the cardiac signal to provide an optimized cardiac signal.

In another example, aspects of this disclosure relate to a computing apparatus that includes one or more processors and operatively couplable to a sensing apparatus. The computing apparatus configured to monitor cardiac electrical activity using the sensing apparatus to generate a cardiac signal over time, detect a pacing artifact in the cardiac signal, and determine to account for the pacing artifact when using the cardiac signal based on at least one pacing artifact characteristic of the pacing artifact in the cardiac signal. The computing apparatus is further configured to account for the pacing artifact when using the cardiac signal if it is determined to account for the pacing artifact. Accounting for the pacing artifact includes removing the pacing artifact from the cardiac signal between a first data point at time T1 and a second data point at time T2 of the cardiac signal and replacing the removed pacing artifact in the cardiac signal with replacement data points disposed along a straight line that extends between the first data point and the second data point of the cardiac signal to provide an optimized cardiac signal.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic diagram of one embodiment of a system that includes an electrode apparatus, a display apparatus, and a computing apparatus.

FIG. 2 is a schematic perspective view of one embodiment of an external electrode apparatus for measuring torso-surface potentials.

FIG. 3 is a schematic perspective view of another embodiment of an external electrode apparatus for measuring torso-surface potentials.

FIG. 4 is a flowchart of one embodiment of a method for mitigating a pacing artifact.

FIG. 5 is a flowchart of one embodiment of a method for accounting for a pacing a pacing artifact in the method of FIG. 4.

FIG. 6 is a graph of voltage versus time of one embodiment of a cardiac signal that includes a benign pacing artifact.

FIG. 7 is a graph of voltage versus time of one embodiment of a cardiac signal that includes an adverse pacing artifact.

FIG. 8 is a graph of voltage versus time of the cardiac signal of FIG. 7 after the signal has been sampled by a wide bandpass filter.

FIG. 9 is a graph of voltage versus time of the cardiac signal of FIG. 8 with an adverse pacing artifact removed from the signal and replaced with a desired function.

FIG. 10 is a graph of voltage versus time of a portion of the cardiac signal of FIG. 9.

FIG. 11 is a graph of voltage versus time of the cardiac signal of FIGS. 9-10 after the signal has been sampled by a narrow bandpass filter.

FIG. 12 is a schematic diagram of one embodiment of a system that includes an implantable medical device (IMD).

FIG. 13 is a schematic diagram of the IMD of FIG. 12.

FIG. 14 is a schematic diagram of a portion of a distal end of an electrical lead of the IMD of FIG. 12 disposed in a left ventricle of a heart.

FIG. 15 is a schematic block diagram of the IMD of FIG. 12.

FIG. 16 is a schematic block diagram of IMD (e.g., an implantable pulse generator) circuitry and associated leads employed of the system of FIG. 12.

### DETAILED DESCRIPTION

The techniques of this disclosure generally relate to systems and methods for mitigating artifacts in a cardiac signal of a patient. An exemplary system can include a sensing apparatus and a computing apparatus coupled to the sensing apparatus. The computing apparatus can be configured to detect a pacing artifact in a cardiac signal, determine whether to account for such pacing artifact, and account for the pacing artifact when using the cardiac signal if it is determined to account for the pacing artifact. In one or more embodiments, the computing apparatus can further be configured to account for the pacing artifact by removing the pacing artifact from the cardiac signal and replacing the removed pacing artifact in the cardiac signal with replacement data points in a desired arrangement such as provided by fitting the data points to a desired function.

The exemplary systems and methods described herein may be configured to assist users (e.g., physicians) in configuring cardiac therapy (e.g., cardiac therapy being performed on a patient during and/or after implantation of cardiac therapy apparatus). According to various configurations, the systems and methods herein are configured to be performed in an at least partially autonomous or a fully autonomous manner such that a user (e.g., a physician and/or a patient) may not be involved in the process. One or more embodiments of systems and methods may be described as being noninvasive. For example, one or more embodiments of systems and methods described herein may not need implantable devices such as leads, probes, sensors, catheters, etc. to evaluate and configure the cardiac therapy. Instead, one or more embodiments may use electrical measurements taken noninvasively using, e.g., a plurality of external electrodes attached to the skin of a patient about the patient's torso.

When collecting multichannel waveforms for at least one of optimizing ECG response or for analyzing EKG signals (e.g., using an implantable cardiac monitor), pacing artifacts can undesirably distort the waveforms at the start of the QRS and cause the measurements to be skewed. This is especially an issue with unipolar pacing and larger pacing amplitudes and pulse widths, which can produce much larger pacing artifacts. After the waveforms have been through bandpass filtering, pacing artifacts can be more difficult to separate from the intrinsic signal due to the time domain smearing of the signal.

One or more embodiments of systems and methods described herein can be utilized to remove such pacing artifacts while maintaining the integrity of the intrinsic signal. For example, one exemplary technique can include monitoring cardiac electrical activity to generate a cardiac signal over time. In one or more embodiments, the cardiac signal can be sampled by a wide bandpass filter so that the frequency content of one or more pacing artifacts is minimally filtered. One or more pacing artifacts can be detected in the cardiac signal, which can either be done in hardware or in software, for example, by detecting a fast first derivative of the voltage over time (dV/dt) in the cardiac signal. Once the location of the pacing artifact has been detected, a determination of whether to account for such artifacts can be made. If it is determined that such accounting will be made, then pacing artifact can be removed from the cardiac signal and replaced with one or more data points. In one or more embodiments, such data points can be fitted to a desired function that is inserted into the cardiac signal in place of the removed pacing artifact. The cardiac signal can optionally be passed through a narrow bandpass filter for CRT pacing optimization measurements. Due to the short length of the replaced segment of the cardiac signal, the pacing artifact can be cleanly removed from the intrinsic signal.

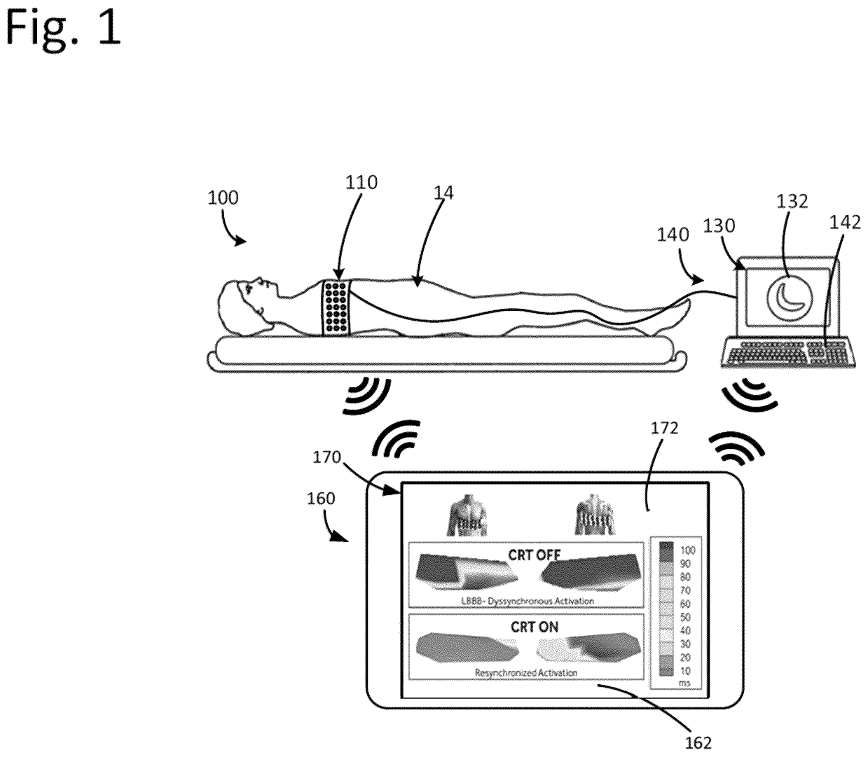

Illustrative systems and methods shall be described with reference to FIGS. 1-16. It will be apparent to one skilled in the art that elements or processes from one embodiment may be used in combination with elements or processes of the other embodiments, and that the possible embodiments of such systems and methods using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure; although, certain timings, one or more shapes and/or sizes, or types of elements, may be advantageous over others.

A plurality of ECG signals (e.g., torso-surface potentials) may be measured, or monitored, using a plurality of external electrodes positioned about the surface, or skin, of a patient. The ECG signals may be used to evaluate a patient's cardiac condition and configure cardiac therapy such as, e.g., cardiac therapy provide by an IMD performing CRT. As described herein, the ECG signals may be gathered or obtained noninvasively since, e.g., implantable electrodes may not be used to measure the ECG signals. Further, the ECG signals may be used to determine cardiac electrical activation times, which may be used to generate various metrics (e.g., electrical heterogeneity information) that may be used by a user (e.g., a physician) to optimize one or more settings, or parameters, of cardiac therapy (e.g., pacing therapy) such as CRT.

Various illustrative systems, methods, and graphical user interfaces may be configured to use an electrode apparatus including external electrodes, a display apparatus, and computing apparatus to noninvasively assist a user (e.g., the physician) in the evaluation of cardiac health and/or the configuration (e.g., optimization) of cardiac therapy. An illustrative system 100 including electrode apparatus 110, computing apparatus 140, and a remote computing device 160 is depicted in FIG. 1.

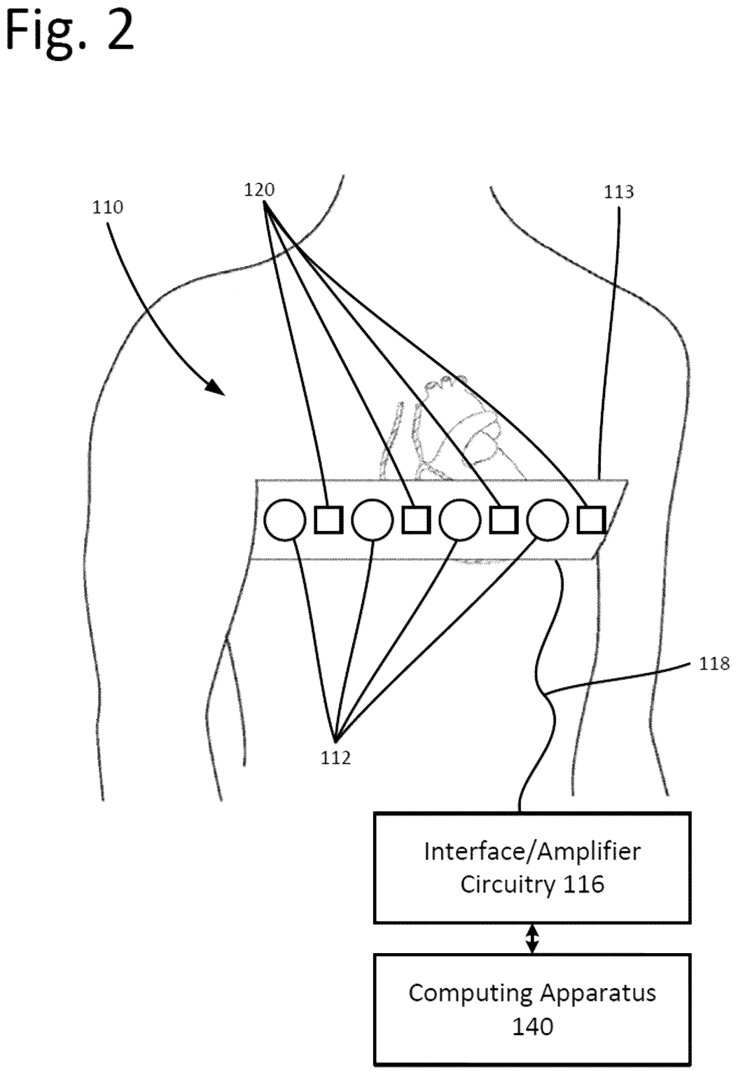

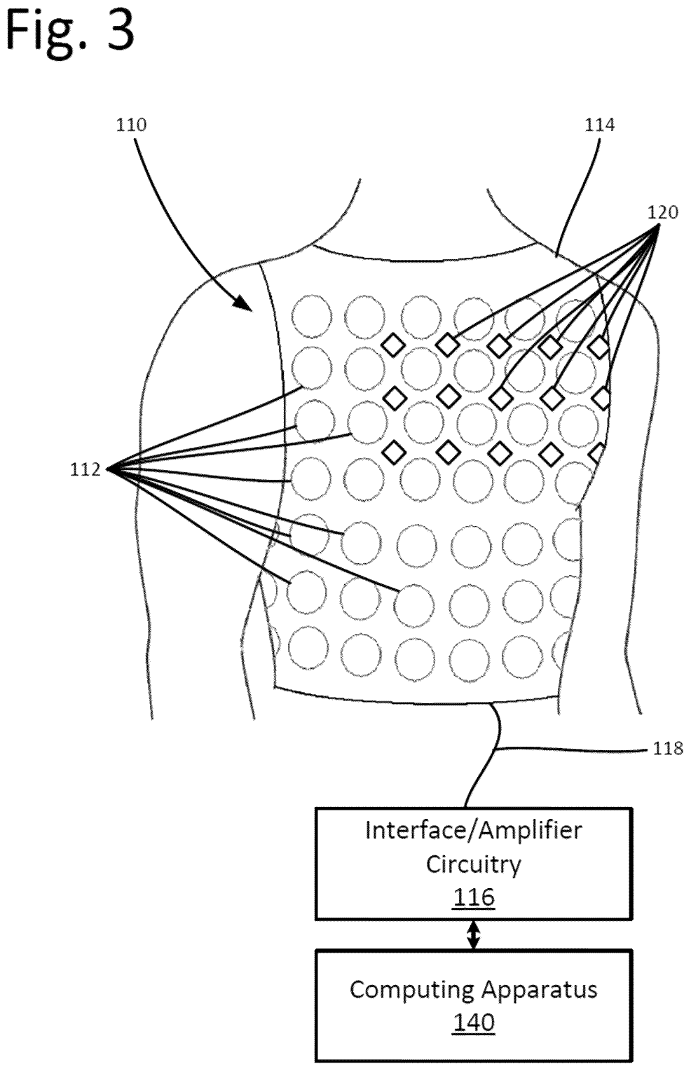

The electrode apparatus 110 as shown includes a plurality of electrodes incorporated, or included, within a band wrapped around the chest, or torso, of a patient 14. The electrode apparatus 110 is operatively coupled to the computing apparatus 140 (e.g., through one or wired electrical connections, wirelessly, etc.) to provide electrical signals from each of the electrodes to the computing apparatus 140 for analysis, evaluation, pacing artifact detection, etc. Illustrative electrode apparatuses may be described in U.S. Pat. No. 9,320,446 entitled BIOELECTRIC SENSOR DEVICE AND METHODS, filed Mar. 27, 2014, and issued on Mar. 26, 2016. Further, illustrative electrode apparatus 110 will be described in more detail in reference to FIGS. 2-3.

Although not described herein, the illustrative system 100 may further include an imaging apparatus. The imaging apparatus may be any type of imaging apparatus configured to image, or provide images of, at least a portion of the patient in a noninvasive manner. For example, the imaging apparatus may not use any components or parts that may be located within the patient to provide images of the patient except noninvasive tools such as contrast solution. It is to be understood that the illustrative systems, methods, and interfaces described herein may further use an imaging apparatus to provide noninvasive assistance to a user (e.g., the physician) to locate, or place, one or more pacing electrodes proximate the patient's heart in conjunction with the configuration of cardiac therapy.

For example, the illustrative systems and methods described herein may provide image guided navigation that may be used to navigate leads including electrodes, leadless electrodes, wireless electrodes, catheters, etc., within the patient's body while also providing noninvasive cardiac therapy configuration including determining effective, or optimal, pre-excitation intervals such as A-V and V-V intervals, etc. Illustrative systems and methods that use imaging apparatus and/or electrode apparatus may be described in at least one of U.S. Pat. App. Pub. No. 2014/0371832 to Ghosh published on Dec. 18, 2014; U.S. Pat. App. Pub. No. 2014/0371833 to Ghosh et al. published on Dec. 18, 2014; U.S. Pat. App. Pub. No. 2014/0323892 to Ghosh et al. published on Oct. 30, 2014; or U.S. Pat. App. Pub. No. 2014/0323882 to Ghosh et al. published on Oct. 20, 2014.

Illustrative imaging apparatuses may be configured to capture x-ray images and/or any other alternative imaging modality. For example, the imaging apparatus may be configured to capture images, or image data, using isocentric fluoroscopy, bi-plane fluoroscopy, ultrasound, computed tomography (CT), multi-slice computed tomography (MSCT), magnetic resonance imaging (MRI), high frequency ultrasound (HIFU), optical coherence tomography (OCT), intra-vascular ultrasound (IVUS), two dimensional (2D) ultrasound, three dimensional (3D) ultrasound, four dimensional (4D) ultrasound, intraoperative CT, intraoperative MRI, etc. Further, it is to be understood that the imaging apparatus may be configured to capture a plurality of consecutive images (e.g., continuously) to provide video frame data. In other words, a plurality of images taken over time using the imaging apparatus may provide video frame, or motion picture, data. An exemplary system that employs ultrasound can be found in U.S. Pat. App. Pub. No. 2017/0303840 entitled NONINVASIVE ASSESSMENT OF CARDIAC RESYNCHRONIZATION THERAPY to Stadler et al. Additionally, the images may also be obtained and displayed in two, three, or four dimensions. In more advanced forms, four-dimensional surface rendering of the heart or other regions of the body may also be achieved by incorporating heart data or other soft tissue data from a map or from pre-operative image data captured by MRI, CT, or echocardiography modalities. Image datasets from hybrid modalities, such as positron emission tomography (PET) combined with CT, or single photon emission computer tomography (SPECT) combined with CT, could also provide functional image data superimposed onto anatomical data, e.g., to be used to navigate implantable apparatus to target locations within the heart or other areas of interest.

Systems and/or imaging apparatuses that may be used with the illustrative systems and method described herein are described, e.g., in at least one of U.S. Pat. App. Pub. No. 2005/0008210 to Evron et al. published on Jan. 13, 2005; U.S. Pat. App. Pub. No. 2006/0074285 to Zarkh et al. published on Apr. 6, 2006; U.S. Pat. No. 8,731,642 to Zarkh et al. issued on May 20, 2014; U.S. Pat. No. 8,861,830 to Brada et al. issued on Oct. 14, 2014; U.S. Pat. No. 6,980,675 to Evron et al. issued on Dec. 27, 2005; U.S. Pat. No. 7,286,866 to Okerlund et al. issued on Oct. 23, 2007; U.S. Pat. No. 7,308,297 to Reddy et al. issued on Dec. 11, 2011; U.S. Pat. No. 7,308,299 to Burrell et al. issued on Dec. 11, 2011; U.S. Pat. No. 7,321,677 to Evron et al. issued on Jan. 22, 2008; U.S. Pat. No. 7,346,381 to Okerlund et al. issued on Mar. 18, 2008; U.S. Pat. No. 7,454,248 to Burrell et al. issued on Nov. 18, 2008; U.S. Pat. No. 7,499,743 to Vass et al. issued on Mar. 3, 2009; U.S. Pat. No. 7,565,190 to Okerlund et al. issued on Jul. 21, 2009; U.S. Pat. No. 7,587,074 to Zarkh et al. issued on Sep. 8, 2009; U.S. Pat. No. 7,599,730 to Hunter et al. issued on Oct. 6, 2009; U.S. Pat. No. 7,613,500 to Vass et al. issued on Nov. 3, 2009; U.S. Pat. No. 7,742,629 to Zarkh et al. issued on Jun. 22, 2010; U.S. Pat. No. 7,747,047 to Okerlund et al. issued on Jun. 29, 2010; U.S. Pat. No. 7,778,685 to Evron et al. issued on Aug. 17, 2010; U.S. Pat. No. 7,778,686 to Vass et al. issued on Aug. 17, 2010; U.S. Pat. No. 7,813,785 to Okerlund et al. issued on Oct. 12, 2010; U.S. Pat. No. 7,996,063 to Vass et al. issued on Aug. 9, 2011; U.S. Pat. No. 8,060,185 to Hunter et al. issued on Nov. 15, 2011; or U.S. Pat. No. 8,401,616 to Verard et al. issued on Mar. 19, 2013.

The computing apparatus 140 and the remote computing device 160 may each include display apparatus 130, 170, respectively, that may be configured to display and analyze data such as, e.g., electrical signals (e.g., electrocardiogram data), electrical activation times, electrical heterogeneity information, etc. For example, one cardiac cycle, or one heartbeat, of a plurality of cardiac cycles, or heartbeats, represented by the electrical signals collected or monitored by the electrode apparatus 110 may be analyzed and evaluated for one or more metrics including activation times and electrical heterogeneity information that may be pertinent to the therapeutic nature of one or more parameters related to cardiac therapy such as, e.g., pacing parameters, lead location, etc. Prior to analysis for one or more metrics, such signals may be analyzed for pacing artifacts, and any pacing artifacts may be mitigated as will be described further herein. More specifically, for example, the QRS complex of a single cardiac cycle may be evaluated for one or more metrics such as, e.g., QRS onset, QRS offset, QRS peak, electrical heterogeneity information (EHI), electrical activation times referenced to earliest activation time, left ventricular or thoracic standard deviation of electrical activation times (LVED), standard deviation of activation times (SDAT), average left ventricular or thoracic surrogate electrical activation times (LVAT), QRS duration (e.g., interval between QRS onset to QRS offset), difference between average left surrogate and average right surrogate activation times, relative or absolute QRS morphology, difference between a higher percentile and a lower percentile of activation times (higher percentile may be 90%, 80%, 75%, 70%, etc. and lower percentile may be 10%, 15%, 20%, 25% and 30%, etc.), other statistical measures of central tendency (e.g., median or mode), dispersion (e.g., mean deviation, standard deviation, variance, interquartile deviations, range), etc. Further, each of the one or more metrics may be location specific. For example, some metrics may be computed from signals recorded, or monitored, from electrodes positioned about a selected area of the patient such as, e.g., the left side of the patient, the right side of the patient, etc.

In one or more embodiments, one or both of the computing apparatus 140 and the remote computing device 160 may be at least one of a server, a personal computer, a tablet computer, a mobile device, or a cellular telephone. The computing apparatus 140 may be configured to receive input from input apparatus 142 (e.g., a keyboard) and transmit output to the display apparatus 130, and the remote computing device 160 may be configured to receive input from input apparatus 162 (e.g., a touchscreen) and transmit output to the display apparatus 170. One or both of the computing apparatus 140 and the remote computing device 160 may include data storage that may allow for access to processing programs or routines and/or one or more other types of data, e.g., for analyzing a plurality of electrical signals captured by the electrode apparatus 110, for detecting and mitigating pacing artifacts in the electrical signals, for determining QRS onsets, QRS offsets, medians, modes, averages, peaks or maximum values, valleys or minimum values, for determining electrical activation times, for driving a graphical user interface configured to noninvasively assist a user in configuring one or more pacing parameters, or settings, such as, e.g., pacing rate, atrial pacing rate ventricular pacing rate, A-V interval, V-V interval, pacing pulse width, pacing vector, multipoint pacing vector (e.g., left ventricular vector quad lead), pacing voltage, pacing configuration (e.g., biventricular pacing, right ventricle only pacing, left ventricle only pacing, etc.), and arrhythmia detection and treatment, rate adaptive settings and performance, etc.

The computing apparatus 140 may be operatively coupled to the input apparatus 142 and the display apparatus 130 to, e.g., transmit data to and from each of the input apparatus 142 and the display apparatus 130, and the remote computing device 160 may be operatively coupled to the input apparatus 162 and the display apparatus 170 to, e.g., transmit data to and from each of the input apparatus 162 and the display apparatus 170. For example, the computing apparatus 140 and the remote computing device 160 may be electrically coupled to the input apparatus 142, 162 and the display apparatus 130, 170 using, e.g., analog electrical connections, digital electrical connections, wireless connections, bus-based connections, network-based connections, internet-based connections, etc. As described further herein, a user may provide input to the input apparatus 142, 162 to view and/or select one or more pieces of configuration information related to the cardiac therapy delivered by a cardiac therapy apparatus such as, e.g., an implantable medical device.

Although as depicted the input apparatus 142 is a keyboard and the input apparatus 162 is a touchscreen, it is to be understood that the input apparatuses 142, 162 may include any apparatus capable of providing input to the computing apparatus 140 and the computing device 160 to perform the functionality, methods, and/or logic described herein. For example, the input apparatuses 142, 162 may include a keyboard, a mouse, a trackball, a touchscreen (e.g., capacitive touchscreen, a resistive touchscreen, a multi-touch touchscreen, etc.), etc. Likewise, the display apparatuses 130, 170 may include any apparatus capable of displaying information to a user, such as a graphical user interface 132, 172 including electrode status information, pacing artifact information, graphical maps of electrical activation, a plurality of signals for the external electrodes over one or more heartbeats, QRS complexes, various cardiac therapy scenario selection regions, various rankings of cardiac therapy scenarios, various pacing parameters, EHI, textual instructions, graphical depictions of anatomy of a human heart, images or graphical depictions of the patient's heart, graphical depictions of locations of one or more electrodes, graphical depictions of a human torso, images or graphical depictions of the patient's torso, graphical depictions or actual images of implanted electrodes and/or leads, etc. Further, the display apparatuses 130, 170 may include a liquid crystal display, an organic light-emitting diode screen, a touchscreen, a cathode ray tube display, etc.

The processing programs or routines stored and/or executed by the computing apparatus 140 and the remote computing device 160 may include programs or routines for pacing artifact detection, determining to account for pacing artifacts, accounting for pacing artifacts (e.g., mitigating such pacing artifacts), filtering, computational mathematics, matrix mathematics, decomposition algorithms, compression algorithms (e.g., data compression algorithms), calibration algorithms, image construction algorithms, signal processing algorithms (e.g., various filtering algorithms, Fourier transforms, fast Fourier transforms, etc.), standardization algorithms, comparison algorithms, vector mathematics, or any other processing used to implement one or more illustrative methods and/or processes described herein. Data stored and/or used by the computing apparatus 140 and the remote computing device 160 may include, for example, electrical signal/waveform data from the electrode apparatus 110 (e.g., a plurality of QRS complexes), pacing artifact thresholds, IMD recharge times, pacing artifact characteristics, electrical activation times from the electrode apparatus 110, cardiac sound/signal/waveform data from acoustic sensors, graphics (e.g., graphical elements, icons, buttons, windows, dialogs, pull-down menus, graphic areas, graphic regions, 3D graphics, etc.), graphical user interfaces, results from one or more processing programs or routines employed according to the disclosure herein (e.g., electrical signals, electrical heterogeneity information, etc.), or any other data that may be used for carrying out the one and/or more processes or methods described herein.

In one or more embodiments, the illustrative systems, methods, and interfaces may be implemented using one or more computer programs executed on programmable computers, such as computers that include, for example, processing capabilities, data storage (e.g., volatile or non-volatile memory and/or storage elements), input devices, and output devices. Program code and/or logic described herein may be applied to input data to perform functionality described herein and generate desired output information. The output information may be applied as input to one or more other devices and/or methods as described herein or as would be applied in a known fashion.

The one or more programs used to implement the systems, methods, and/or interfaces described herein may be provided using any programmable language, e.g., a high-level procedural and/or object orientated programming language that is suitable for communicating with a computer system. Any such programs may, for example, be stored on any suitable device, e.g., a storage media, which is readable by a general or special purpose program running on a computer system (e.g., including processing apparatus) for configuring and operating the computer system when the suitable device is read for performing the procedures described herein. In other words, in one or more embodiments, the illustrative systems, methods, and interfaces may be implemented using a computer readable storage medium, configured with a computer program, where the storage medium so configured causes the computer to operate in a specific and predefined manner to perform functions described herein. Further, in one or more embodiments, the illustrative systems, methods, and interfaces may be described as being implemented by logic (e.g., object code) encoded in one or more non-transitory media that includes code for execution and, when executed by a processor or processing circuitry, is operable to perform operations such as the methods, processes, and/or functionality described herein.

The computing apparatus 140 and the remote computing device 160 may be, for example, any fixed or mobile computer system (e.g., a controller, a microcontroller, a personal computer, minicomputer, tablet computer, etc.). The exact configurations of the computing apparatus 140 and the remote computing device 160 are not limiting, and essentially any device capable of providing suitable computing capabilities and control capabilities (e.g., signal analysis, mathematical functions such as medians, modes, averages, maximum value determination, minimum value determination, slope determination, minimum slope determination, maximum slope determination, graphics processing, etc.) may be used. As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by the computing apparatus 140 and the remote computing device 160 described herein. Also, as described herein, a file in user-readable format may be any representation of data (e.g., ASCII text, binary numbers, hexadecimal numbers, decimal numbers, graphically, etc.) presentable on any medium (e.g., paper, a display, etc.) readable and/or understandable by a user.

In view of the above, it will be readily apparent that the functionality as described in one or more embodiments according to the present disclosure may be implemented in any manner as would be known to one skilled in the art. As such, the computer language, the computer system, or any other software/firmware/hardware which is to be used to implement the processes described herein shall not be limiting on the scope of the systems, processes, or programs (e.g., the functionality provided by such systems, processes, or programs) described herein.

The illustrative electrode apparatus 110 may be configured to measure body-surface potentials of a patient 14 and, more particularly, torso-surface potentials of the patient. As shown in FIG. 2, the illustrative electrode apparatus 110 may include a set, or array, of external electrodes 112, a strap 113, and interface/amplifier circuitry 116. The electrodes 112 may be attached, or coupled, to the strap 113, and the strap may be configured to be wrapped at least partially around the torso of the patient 14 such that the electrodes surround the patient's heart. As further illustrated, the electrodes 112 may be positioned around the circumference of the patient 14, including the posterior, lateral, posterolateral, anterolateral, and anterior locations of the torso of a patient.

The illustrative electrode apparatus 110 may be further configured to measure, or monitor, sounds from at least one or both of the heart or the patient 14. As shown in FIG. 2, the illustrative electrode apparatus 110 may include a set, or array, of acoustic sensors 120 attached, or coupled, to the strap 113. The strap 113 may be configured to be wrapped at least partially around the torso of a patient 14 such that the acoustic sensors 120 surround the patient's heart. As further illustrated, the acoustic sensors 120 may be positioned around the circumference of the patient 14, including the posterior, lateral, posterolateral, anterolateral, and anterior locations of the torso of the patient.

Further, the electrodes 112 and the acoustic sensors 120 may be electrically connected to interface/amplifier circuitry 116 via wired connection 118. The interface/amplifier circuitry 116 may be configured to amplify the signals from the electrodes 112 and the acoustic sensors 120 and provide the signals to one or both of the computing apparatus 140 and the remote computing device 160. Other illustrative systems may use a wireless connection to transmit the signals sensed by electrodes 112 and the acoustic sensors 120 to the interface/amplifier circuitry 116 and, in turn, to one or both of the computing apparatus 140 and the remote computing device 160, e.g., as channels of data. In one or more embodiments, the interface/amplifier circuitry 116 may be electrically coupled to the computing apparatus 140 using, e.g., analog electrical connections, digital electrical connections, wireless connections, bus-based connections, network-based connections, internet-based connections, etc.

Although in the example of FIG. 2 the electrode apparatus 110 includes a strap 113, in other examples any of a variety of mechanisms, e.g., tape or adhesives, may be employed to aid in the spacing and placement of electrodes 112 and the acoustic sensors 120. In one or more embodiments, the strap 113 may include an elastic band, strip of tape, or cloth. Further, in one or more embodiments, the strap 113 may be part of, or integrated with, a piece of clothing such as, e.g., a t-shirt. In one or more embodiments, the electrodes 112 and the acoustic sensors 120 may be placed individually on the torso of the patient 14. Further, in one or more embodiments, one or both of the electrodes 112 (e.g., arranged in an array) and the acoustic sensors 120 (e.g., also arranged in an array) may be part of, or located within, patches, vests, and/or other manners of securing the electrodes 112 and the acoustic sensors 120 to the torso of the patient 14. Still further, in other examples, one or both of the electrodes 112 and the acoustic sensors 120 may be part of, or located within, two sections of material or two patches. One of the two patches may be located on the anterior side of the torso of the patient 14 (to, e.g., monitor electrical signals representative of the anterior side of the patient's heart, measure surrogate cardiac electrical activation times representative of the anterior side of the patient's heart, monitor or measure sounds of the anterior side of the patient, etc.) and the other patch may be located on the posterior side of the torso of the patient 14 (to, e.g., monitor electrical signals representative of the posterior side of the patient's heart, measure surrogate cardiac electrical activation times representative of the posterior side of the patient's heart, monitor or measure sounds of the posterior side of the patient, etc.). And still further, in one or more embodiments, one or both of the electrodes 112 and the acoustic sensors 120 may be arranged in a top row and bottom row that extend from the anterior side of the patient 14 across the left side of the patient to the posterior side of the patient. Yet still further, in one or more embodiments, one or both of the electrodes 112 and the acoustic sensors 120 may be arranged in a curve around the armpit area and may have an electrode/sensor-density that is less dense on the right thorax than the other remaining areas.

The electrodes 112 may be configured to surround the heart of the patient 14 and record, or monitor, the electrical signals associated with the depolarization and repolarization of the heart after the signals have propagated through the torso of a patient 14. Each of the electrodes 112 may be used in a unipolar configuration to sense the torso-surface potentials that reflect the cardiac signals. The interface/amplifier circuitry 116 may also be coupled to a return or indifferent electrode (not shown) that may be used in combination with each electrode 112 for unipolar sensing.

Any suitable number of electrodes 112 may be utilized for sensing electrical signals. In one or more embodiments, about 3 to 5 electrodes 112 may be utilized. In one or more embodiments, about 12 to about 50 electrodes 112 and about 12 to about 50 acoustic sensors 120 may be spatially distributed around the torso of a patient. Other configurations may have more or fewer electrodes 112 and more or fewer acoustic sensors 120. It is to be understood that the electrodes 112 and acoustic sensors 120 may not be arranged or distributed in an array extending all the way around or completely around the patient 14. Instead, the electrodes 112 and acoustic sensors 120 may be arranged in an array that extends only part of the way or partially around the patient 14. For example, the electrodes 112 and acoustic sensors 120 may be distributed on the anterior, posterior, and left sides of the patient with less or no electrodes and acoustic sensors proximate the right side (including posterior and anterior regions of the right side of the patient).

The computing apparatus 140 may record and analyze the torso-surface potential signals sensed by electrodes 112 and the sound signals sensed by the acoustic sensors 120, which are amplified/conditioned by the interface/amplifier circuitry 116. The computing apparatus 140 may be configured to analyze the electrical signals from the electrodes 112 to provide electrocardiogram (ECG) signals, information, or data from the patient's heart as will be further described herein. The computing apparatus 140 may be configured to detect and account for pacing artifacts in such signals as will be further described herein. The computing apparatus 140 may be configured to analyze the electrical signals from the acoustic sensors 120 to provide sound signals, information, or data from the patient's body and/or devices implanted therein (such as a left ventricular assist device).

Additionally, the computing apparatus 140 and the remote computing device 160 may be configured to provide graphical user interfaces 132, 172 depicting various information related to the electrode apparatus 110 and the data gathered, or sensed, using the electrode apparatus 110. For example, the graphical user interfaces 132, 172 may depict ECGs including QRS complexes obtained using the electrode apparatus 110 and sound data including sound waves obtained using the acoustic sensors 120 as well as other information related thereto. Illustrative systems and methods may noninvasively use the electrical information collected using the electrode apparatus 110 and the sound information collected using the acoustic sensors 120 to evaluate a patient's cardiac health and to evaluate and configure cardiac therapy being delivered to the patient.

Further, the electrode apparatus 110 may further include reference electrodes and/or drive electrodes to be, e.g., positioned about the lower torso of the patient 14, that may be further used by the system 100. For example, the electrode apparatus 110 may include three reference electrodes, and the signals from the three reference electrodes may be combined to provide a reference signal. Further, the electrode apparatus 110 may use three caudal reference electrodes (e.g., instead of standard references used in a Wilson Central Terminal) to get a “true” unipolar signal with less noise from averaging three caudally located reference signals.

FIG. 3 illustrates another illustrative electrode apparatus 110 that includes a plurality of electrodes 112 configured to surround the heart of the patient 14 and record, or monitor, the electrical signals associated with the depolarization and repolarization of the heart after the signals have propagated through the torso of the patient 14 and a plurality of acoustic sensors 120 configured to surround the heart of the patient 14, and record, or monitor, the sound signals associated with the heart after the signals have propagated through the torso of the patient. The electrode apparatus 110 may include a vest 114 upon which the plurality of electrodes 112 and the plurality of acoustic sensors 120 may be attached, or to which the electrodes 112 and the acoustic sensors 120 may be coupled. In one or more embodiments, the plurality, or array, of electrodes 112 may be used to collect electrical information such as, e.g., surrogate electrical activation times. Similar to the electrode apparatus 110 of FIG. 2, the electrode apparatus 110 of FIG. 3 may include interface/amplifier circuitry 116 electrically coupled to each of the electrodes 112 and the acoustic sensors 120 through a wired connection 118 and be configured to transmit signals from the electrodes 112 and the acoustic sensors 120 to computing apparatus 140. As illustrated, the electrodes 112 and the acoustic sensors 120 may be distributed over the torso of a patient 14, including, for example, the posterior, lateral, posterolateral, anterolateral, and anterior locations of the torso of a patient 14.

The vest 114 may be formed of fabric with the electrodes 112 and the acoustic sensors 120 attached to the fabric. The vest 114 may be configured to maintain the position and spacing of electrodes 112 and the acoustic sensors 120 on the torso of the patient 14. Further, the vest 114 may be marked to assist in determining the location of the electrodes 112 and the acoustic sensors 120 on the surface of the torso of the patient 14. In one or more embodiments, about 25 to about 256 electrodes 112 and about 25 to about 256 acoustic sensors 120 may be distributed around the torso of the patient 14, though other configurations may have more or fewer electrodes 112 and more or fewer acoustic sensors 120.

The illustrative systems and methods may be used to provide noninvasive assistance to a user in the evaluation of a patient's cardiac health and/or evaluation and configuration of cardiac therapy being presently delivered to the patient (e.g., by an implantable medical device delivering pacing therapy, by an LVAD, etc.). Further, it is to be understood that the computing apparatus 140 and the remote computing device 160 may be operatively coupled to each other in a plurality of different ways to perform, or execute, the functionality described herein. For example, in the embodiment depicted, the computing apparatus 140 may be wirelessly operably coupled to the remote computing device 160 as depicted by the wireless signal lines emanating therebetween. Additionally, as opposed to wireless connections, one or more of the computing apparatus 140 and the remoting computing device 160 may be operably coupled through one or more wired electrical connections.

According to embodiments described herein, the illustrative system 100, which may be referred to as an ECG belt system, may be used with cardiac therapy systems and devices (e.g., CRT pacing devices) to calculate various metrics related to the cardiac health of a patient (e.g., the standard deviation of activation times (SDAT)) across one or more cardiac cycles (or heart beats), and in particular, based on activation times or other data gathered during each QRS event of the cardiac cycle (heart beat). According to various embodiments, the illustrative system 100 may be used to calculate, or generate, electrical heterogeneity information such as, e.g., SDAT, of cardiac cycles during delivery of CRT (e.g., the SDAT for cardiac cycles where CRT paces are delivered). For example, the illustrative system 100 may be used to calculate electrical heterogeneity information for cardiac cycles during biventricular and/or left ventricular pacing. Further, embodiments described herein may be used to evaluate a patient's cardiac health and/or non-CRT pacing. If electrical heterogeneity information is inaccurate, the output of the illustrative system 100 could be misleading, which could potentially affect lead placement (e.g., an implantable lead not being placed at an optimal spot) and/or optimal device programming. For example, if the SDAT is inaccurate, the SDAT may be artificially low, which may cause a clinician to not relocate a currently positioned lead as opposed to repositioning the lead to obtain a better response.

Pacing devices may introduce pacing artifacts that can distort cardiac signals generated from electrical activity monitored by an electrode apparatus. Such distorted cardiac signals may result in the generation of inaccurate electrical heterogeneity information. For example, a pacing artifact with a pacing spike (e.g., a pacing spike that exceeds a given threshold) may cause signal ripple before and after the pacing artifact that distorts a QRS event of the cardiac cycle. Further, for example, a pacing artifact with a recharge time (e.g., a recharge time that exceeds a threshold) may distort the cardiac signals (e.g., because such long pacing recharge times may get through, or slip through, low pass filters in the system).

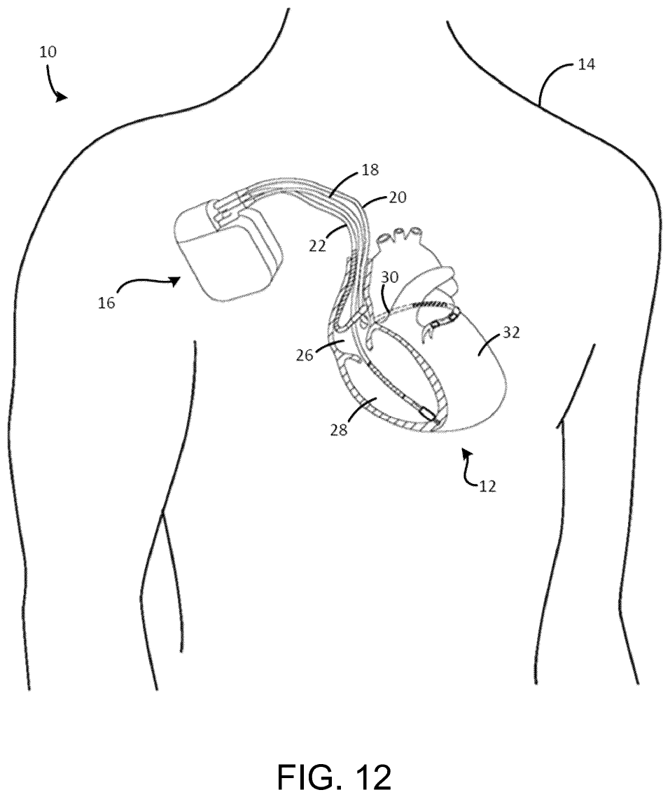

In one or more embodiments, a computing apparatus including a processing apparatus, or one or more processors, can detect and account for pacing artifacts capable of distorting cardiac signals. Pacing artifacts may be detected in body-surface potentials of a patient measured or monitored using external electrode apparatus as described herein. Such pacing artifacts may be introduced to body-surface potentials of a patient by a pacing device (e.g., an implantable medical device 16 of FIG. 12); however, such pacing artifacts may be detected without the use of the pacing device as will be described herein. In other words, the ECG belt and computing apparatus may not communicate with the pacing device to aid in detection of pacing artifacts.

Each of such detected pacing artifacts may include a pacing spike and a recharge time that may be used to determine the extent and duration of pacing artifacts. In other words, one or both of a packing spike and a recharge time may be associated with, or correspond to, a detected pacing artifact or artifacts. As described herein, the extent and duration of a pacing artifact may be determined, and once the extent and duration of a pacing artifact is determined, the computing apparatus may account for the pacing artifact (e.g., mitigate the pacing artifact). To account for the pacing artifact, the computing apparatus may be configured to remove or filter out the pacing artifact (e.g., pacing spike), alert a user to the pacing artifact, provide an error message (e.g., via a graphical user interface) if no pacing spikes are detected while recording paced events (which may indicate intrinsic activity or premature complexes), etc.

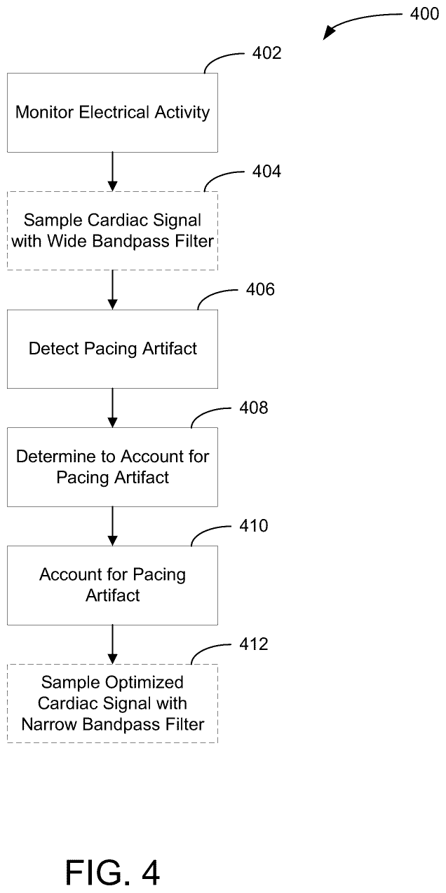

FIG. 4 is a flowchart of one embodiment of a method or technique 400 for accounting for pacing artifacts. The method 400 may utilize, or be carried out using, an electrode apparatus (e.g., electrode apparatus 110) and a computing apparatus (e.g., computing apparatus 140). Electrical activity may be monitored using one or more external electrodes of the electrode apparatus 110 to generate a cardiac signal over time at 402. For example, the electrical activity may be monitored over a plurality of cardiac cycles or heartbeats. Further, the electrical activity may be monitored for a selected period of time such as, e.g., five seconds, or continuously. The electrode apparatus 110 may measure body-surface potentials of the patient 14 and generate the cardiac signal based on the body-surface potentials. The cardiac signal may include pacing artifacts introduced by a pacing device (e.g., pacing device 16 of FIG. 12) that is delivering pacing therapy to the patient 14.

At 404, the cardiac signal can optionally be sampled with a wide bandpass filter using any suitable technique. The wide bandpass filter can have any suitable bandwidth. In one or more embodiments, the wide bandpass filter can have a bandwidth of at least 400 Hz. In one or more embodiments, the bandwidth of the wide bandpass filter can be no greater than 2 kHz.

A pacing artifact may be detected in the cardiac signal at 406 using any suitable technique. In one or more embodiments, the pacing artifact can be detected by determining one or more pacing artifact characteristics of the pacing artifact such as, for example, an onset time, a duration, a pacing spike amplitude, a pacing recharge duration, a first derivative of the voltage of the cardiac signal relative to time (dV/dt), etc.

It may be determined to account for the pacing artifact when using the cardiac signal based on at least one pacing artifact characteristic of the pacing artifact in the cardiac signal at 408. Pacing artifact characteristics of the pacing artifact may indicate whether or not the pacing artifact can distort the one or more cardiac signals enough to result in inaccurate electrical heterogeneity information. Accordingly, determining to account for the pacing artifact based on at least one pacing artifact characteristic can allow disruptive pacing artifacts to be accounted for while ignoring pacing artifacts that do not impact the accuracy of electrical heterogeneity information.

Determining to account for the pacing artifact 408 may include comparing the at least one pacing artifact characteristic to a threshold. Such thresholds may include, for example, a first derivative threshold of the first derivative of voltage relative to time (dV/dt) of the cardiac signal, a spike amplitude threshold, a pacing recharge time amplitude, a pacing duration threshold, or other pacing artifact characteristic threshold. For example, a maximum dV/dt may be compared to a first derivative threshold to determine whether to account for the pacing artifact. The first derivative threshold of dV/dt can be at least 0.1 V/sec and no greater than 1000 V/sec. In one or more embodiments, the first derivative threshold can be at least 1 V/sec and no greater than 100 V/sec. Further, for example, a maximum spike amplitude of the pacing artifact may be compared to a spike amplitude threshold to determine whether to account for the pacing artifact. The spike amplitude threshold may be within a range of about 0.5 volts to about 6 volts. In one or more embodiments, the spike amplitude threshold may be about 3 volts. Further for example, the pacing recharge time of the pacing artifact may be compared to a pacing recharge time threshold to determine whether to account for the pacing artifact. The pacing recharge time threshold may be based on the specifications of a pacing device. The pacing recharge time threshold may be within a range of about 4 milliseconds to about 8 milliseconds. In one or more embodiments, the pacing recharge time threshold may be about 6.5 milliseconds.

The pacing artifact may be accounted for, or handled, if it is determined to account for the pacing artifact at 410. In one or more embodiments, the determination to account for the pacing artifact 408 can be eliminated, and the pacing artifact can be accounted for at 410 after such pacing artifact has been detect 406. Accounting for the pacing artifact may include one or more of, for example, blanking the one or more cardiac signals, adjusting a detected QRS onset, alerting a user of the pacing artifact, marking the pacing artifact, etc.

In one or more embodiments, accounting for the pacing artifact may include smoothing the portion of a cardiac signal that includes the pacing artifact by removing such portion of the signal (e.g., removing the pacing artifact) or replacing such portion of the signal (e.g., interpolation, flatlining the signal, etc.). Such smoothing may be performed, or carried out, by a computing device (e.g., computing apparatus 140) and/or an amplifier (e.g., amplifier circuitry 116). These techniques for accounting for the pacing artifact are different from the technique of “blanking,” which involves disconnecting an input of a sense amplifier from therapy electrodes to avoid the pacing artifact, or ignoring the sense amplifier output in the digital domain during the occurrence of the artifact such that the artifact does not get passed to the rest of the system. In fact, disconnecting and reconnecting the input of the sense amplifier can create additional artifacts in the cardiac signal.

Removal of the portion of the cardiac signal that includes the pacing artifact may result in a cardiac signal that includes discontinuities. In other words, a cardiac signal with the pacing spike removed may stop at a time about where the pacing artifact began and the cardiac signal with the pacing spike removed may resume at a time about where the pacing artifact ended. Removal of the pacing artifact from the cardiac signal by interpolation may include estimating a function to replace the portion of the cardiac signal that includes the pacing spike based on values of the cardiac signal before and after the pacing artifact. Such estimated function may be substituted for the portion of the cardiac signal that includes the pacing artifact. Removal of the pacing artifact from a cardiac signal by flatlining the signal may include replacing the portion of the cardiac signal that includes the pacing artifact with a single voltage value for each point during the time period that includes the pacing artifact. The single value may be a predetermined value or may be determined based on values of the cardiac signal before or after the pacing artifact.

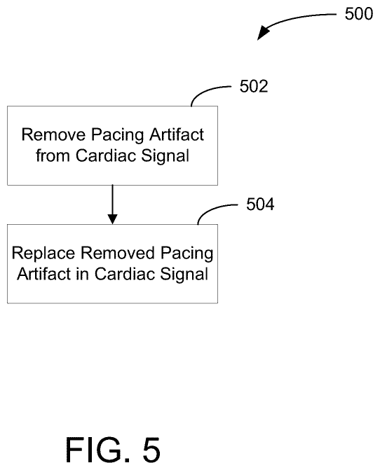

For example, FIG. 5 is a flowchart of one embodiment of a method 500 of accounting for the pacing artifact at 410 of FIG. 4. The method 500 includes removing the pacing artifact from the cardiac signal between a first data point at time T1 and a second data point at time T2 of the cardiac signal at 502. Any suitable technique can be utilized to remove the pacing artifact. An interval between T1 and T2 can be any suitable value. In one or more embodiments, the interval between T1 and T2 is at least 10 milliseconds. In one or more embodiments, the interval between T1 and T2 is no greater than 100 milliseconds, no greater than 50 milliseconds, or no greater than 30 milliseconds.

At 504 the removed pacing artifact can be replaced in the cardiac signal with one or more replacement data points in any suitable arrangement. In one or more embodiments, the removed pacing artifact can be replaced with two or more data points in the cardiac signal that are disposed on or fitted to any suitable function having endpoints at the first data point and the second data point to provide an optimized cardiac signal. In one or more embodiments, the data points can be disposed along a straight line that extends between the first data point and the second data point of the cardiac signal to provide an optimized cardiac signal.

In one or more embodiments, accounting for the pacing artifact may include comparing a position of a pacing spike in the one or more cardiac signals to a detected QRS onset and adjusting the detected QRS onset in response to the detected QRS onset occurring before the position of the pacing spike. In general, pacing spikes precede a QRS onset because pacing caused the heart to beat. However, adverse pacing artifacts can cause QRS onset detection processes to falsely identify a portion of the adverse pacing artifact as the QRS onset. A comparison of the position of the pacing spike to the detected QRS onset can indicate whether the detected QRS onset has been erroneously identified. If the detected QRS onset occurs before the pacing spike (e.g., a peak of the pacing spike), the QRS onset can be flagged and/or adjusted. To adjust the QRS onset, QRS onset determination algorithms can be repeated while excluding the portion of the cardiac signal corresponding to a time period of the pacing artifact in the one or more cardiac signals. Accordingly, the systems and methods described herein can correct erroneous QRS onset detection caused by adverse pacing artifacts.

Returning to FIG. 4, accounting for the pacing artifact at 410 may include alerting a user of the pacing artifact. Alerting a user of the pacing artifact may include providing visual or audible indicators using a computing apparatus (e.g., computing apparatus 140) or remote computing device (e.g., remote computing device 160) that indicate to a user that a pacing artifact has distorted the one or more cardiac signals. For example, an audible alarm may be played by one or more audio devices of the computing apparatus or remote computing device. Further, for example, a visual indicator may be provided on a display apparatus (e.g., display apparatus 130) or a graphical user interface (e.g., graphical user interface 132) of the computing apparatus or remote computing device. Visual indicators may include, for example, a blinking light, text, marking, or other visual warning. Marking the pacing artifact in cardiac signal may include indicating on a visual representation of the cardiac signal a position, an activation time, a duration, a pacing spike amplitude, or a pacing recharge time of the pacing artifact. The visual representation of the cardiac signal may be displayed on a display apparatus or graphical user interface of the computing apparatus or remote computing device. Such visual representation may include a graph, plot, sampled data points, or other visual representation of the cardiac signal.

At 412, the optimized cardiac signal can be sampled using any suitable narrow bandpass filter. In one or more embodiments, the narrow bandpass filter can have a bandwidth of at least 10 Hz and no greater than 500 Hz.

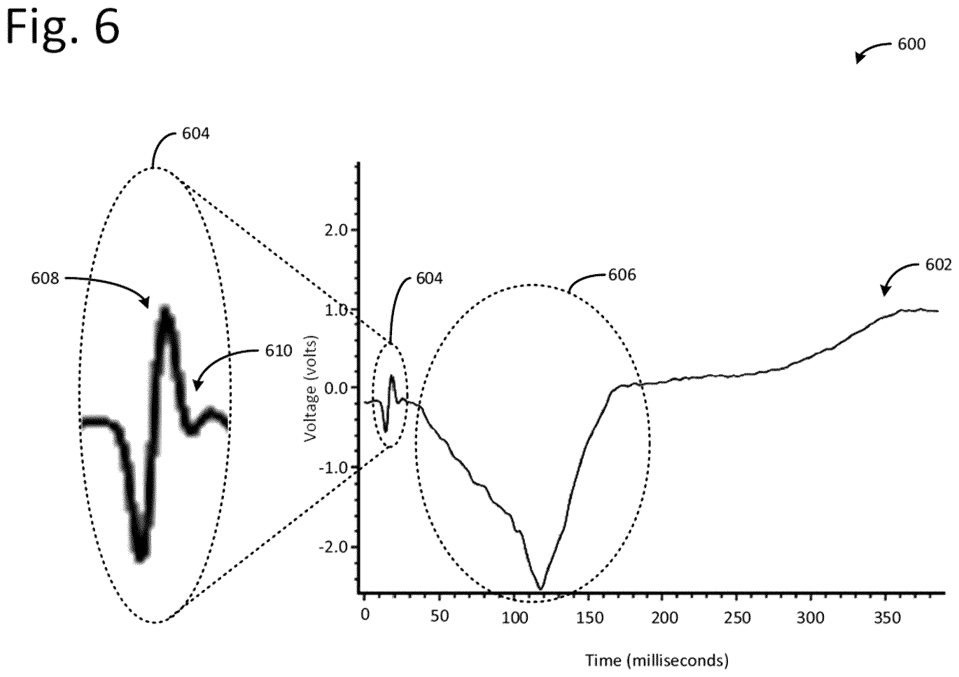

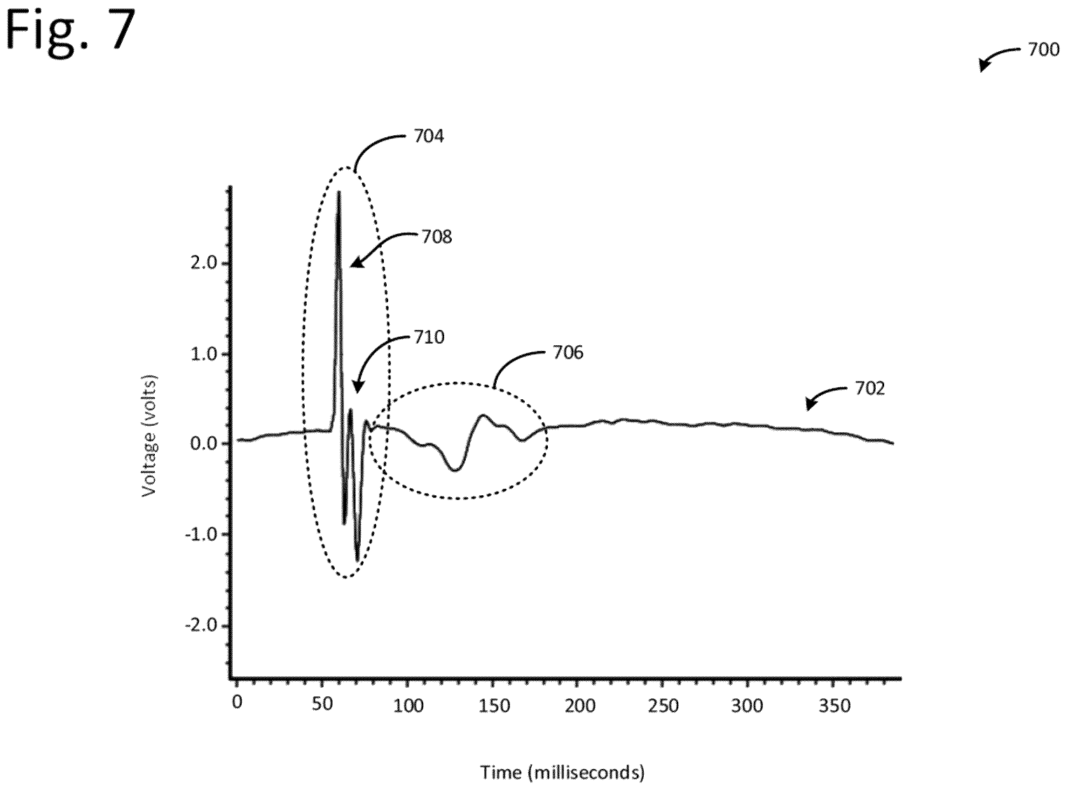

Illustrative cardiac signals generated by electrode apparatuses (e.g., electrode apparatus 110 of FIG. 2 or electrode apparatus 110 of FIG. 3) are depicted in FIGS. 6-11. A cardiac signal 602 that includes a benign pacing artifact 604 is depicted on a graph 600 of FIG. 6 and a cardiac signal 702 that includes an adverse pacing artifact 704 is depicted on a graph 700 in FIG. 7. The cardiac signals 602, 702 of FIGS. 6 and 7 are depicted on graphs 600, 700, respectively, each of which illustrates voltage in volts over time in milliseconds.

The cardiac signal 602 of FIG. 6 provides an example of a benign pacing artifact 604 that may not affect the calculation or generation of electrical heterogeneity information. The cardiac signal 602 includes a QRS complex 606 and the benign pacing artifact 604 includes a pacing spike 608 and a recharge time 610. As shown, the pacing spike 608, the recharge time 610, and any associated distortions are smaller than the QRS complex 606. In other words, an amplitude of the pacing spike 608 is smaller than an amplitude of the QRS complex 606, and a duration of the recharge time 610 is shorter than a duration of the QRS complex 606. Furthermore, the recharge time 610 does not exceed an expected recharge time of a pacing device. Still further, the benign pacing artifact 604 does not impact the QRS complex 606 and may allow for accurate calculation or generation of electrical heterogeneity information without accounting for the pacing artifact. In one or more embodiments, dV/dt of the benign artifact is less than a first derivative threshold.

When applying the method 400 of FIG. 4 to the cardiac signal 602, the benign pacing artifact 604 may not be accounted for. In other words, the benign pacing artifact 604 may be detected and ignored. A computing apparatus (e.g., computing apparatus 140 and/or amplifier circuitry 116) may monitor cardiac electrical activity using one or more external electrodes to generate the cardiac signal 602. The computing apparatus may detect the benign pacing artifact 604 in the cardiac signal 602. However, the computing apparatus may determine not to account for the benign pacing artifact 604 because the benign pacing artifact does not meet any criteria for determining to account for a pacing artifact. Such criteria may include, for example, a first derivative threshold (dV/dt), a spike amplitude threshold, a recharge time threshold, a detected QRS onset earlier than the pacing spike 608, etc. Accordingly, the computing apparatus may not account, or may ignore, the benign pacing artifact 604.

In contrast, the cardiac signal 702 of FIG. 7 provides an example of an adverse pacing artifact 704 that can affect the calculation or generation of electrical heterogeneity information. The cardiac signal 702 includes a QRS complex 706, and the adverse pacing artifact 704 includes a pacing spike 708 and a recharge time 710. As shown, the pacing spike 708 exceeds 2 volts and imparts a ripple to the cardiac signal 702 during the recharge time 710. Additionally, a maximum amplitude of the pacing spike 708 exceeds a maximum amplitude of the QRS complex 706. Still further, the adverse pacing artifact 704 overlaps the QRS complex 706. Further, dV/dt exceeds a first derivative threshold, e.g., the first derivative is greater than a0.1 V/sec threshold.

For any one or more of these reasons (e.g., the maximum amplitude of the pacing spike 708, the recharge time 710, signal ripple caused by the pacing spike, dV/dt, etc.), pacing artifacts such as the adverse pacing artifact 704 can cause inaccurate calculation or generation of electrical heterogeneity. However, if accounted for using the systems and methods described herein, accurate calculation and generation of electrical heterogeneity information can be generated or calculated based on the surface potential signals that cardiac signal 702 was derived from without communication from the pacing device.

When applying the method 400 to the cardiac signal 702, the adverse pacing artifact 704 may be accounted for, and an optimized cardiac signal can be provided. In other words, the adverse pacing artifact 704 may be detected and dealt with in a manner that allows for accurate calculation or generation of electrical heterogeneity. A computing apparatus (e.g., computing apparatus 140 and/or amplifier circuitry 116) may monitor cardiac electrical activity using one or more external electrodes to generate the cardiac signal 702. The computing device may detect the pacing artifact 704 in the cardiac signal 702. The computing apparatus may determine to account for the adverse pacing artifact 704 based on at least one pacing artifact characteristic of the adverse pacing artifact 704 in the cardiac signal 702. Such pacing artifact characteristics may include dV/dt, the maximum amplitude of the pacing spike 708, a duration of the recharge time 710, distortions or ripples caused by the pacing spike 708, etc. The computing apparatus may account for the adverse pacing artifact 704 when using the cardiac signal 702 because it was determined to account for the adverse pacing artifact. Accounting for the adverse pacing artifact 704 may include one or more of, for example, removing the pacing artifact, blanking the one or more cardiac signals, adjusting a detected QRS onset, alerting a user of the pacing artifact, marking the pacing artifact, etc.

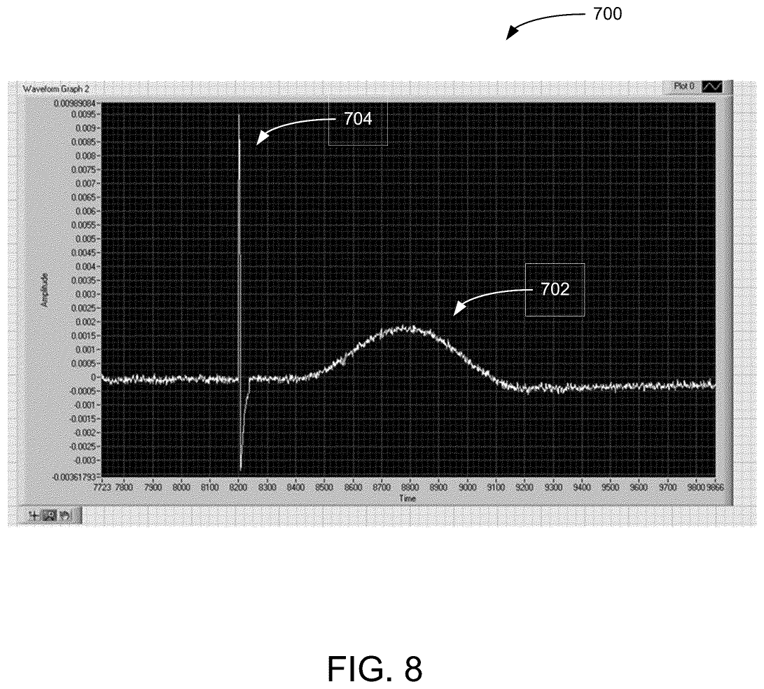

As shown in FIG. 8, the cardiac signal 702 has been sampled with a wide bandpass filter prior to detecting the pacing artifact. Any suitable wide bandpass filter can be utilized. In one or more embodiments, the wide bandpass filter can have a bandwidth of at least 400 Hz. In one or more embodiments, the wide bandpass filter can have a bandwidth of no greater than 2 kHz or no greater than 1 kHz.

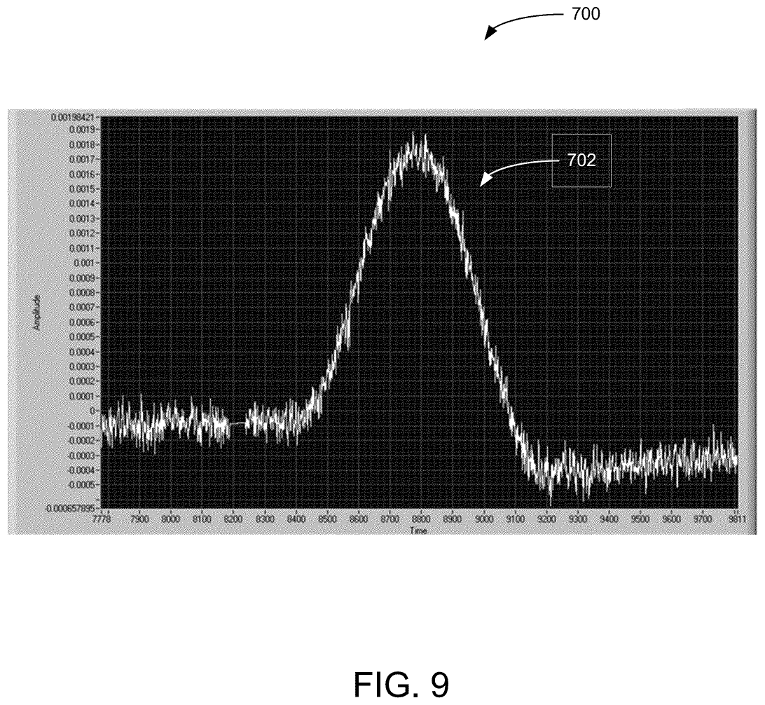

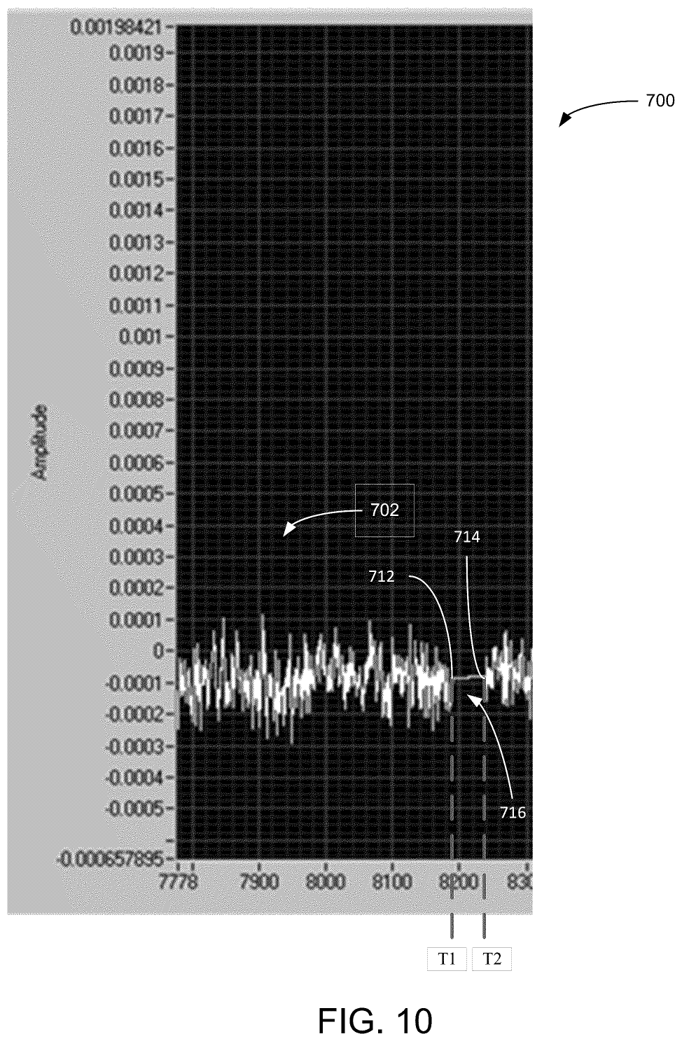

In FIGS. 9-10, the pacing artifact 704 has been removed from the cardiac signal 702 using any suitable technique. As can be seen in FIG. 10, the pacing artifact 704 has been removed between a first data point 712 at time T1 and a second data point 714 at time T2 of the cardiac signal 702. Further, the removed pacing artifact has been replaced in the cardiac signal 702 with replacement data points 716. The replacement data points can include any suitable values. In one or more embodiments, the replacement data points 716 can be fitted or disposed along a function that extends between the first data point 712 and the second data point 714. In one or more embodiments, the replacement data points can be disposed along a straight line that extends between the first data point 712 and the second data point 714 to provide an optimized cardiac signal 702.

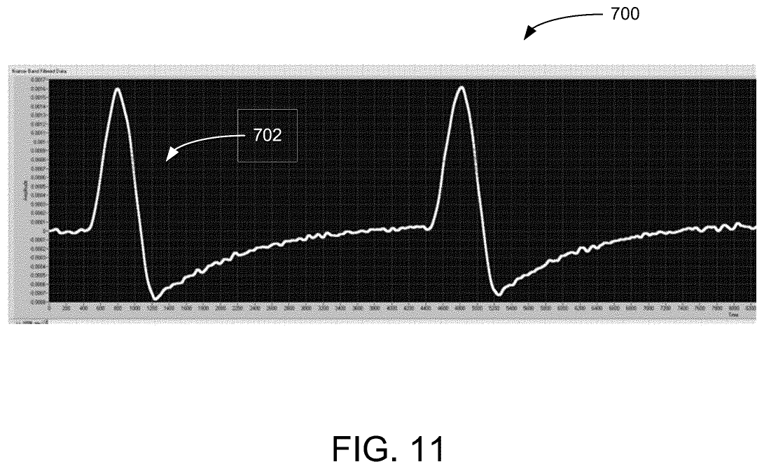

As shown in FIG. 11, the optimized cardiac signal 702 can be sampled with a narrow bandpass filter. Any suitable narrow bandpass filter can be utilized. In one or more embodiments, it may be desirable to sample the cardiac signal 702 after the signal has been optimized as sampling prior to optimization can spread the artifact over a much longer time period, which then may make it more difficult to remove as the frequency content of the artifact more closely resembles the cardiac signal.

Illustrative cardiac therapy systems and devices may be further described herein with reference to FIGS. 12-16 that may utilizes the illustrative systems, interfaces, methods, and processes described herein with respect to FIGS. 1-11.

FIG. 12 is a conceptual diagram illustrating an illustrative therapy system 10 that may be used to deliver pacing therapy to the patient 14. Patient 14 may, but not necessarily, be a human. The therapy system 10 may include any suitable implantable medical device 16 (IMD), which may be coupled to leads 18, 20, 22. The IMD 16 may be, e.g., an implantable pacemaker, cardioverter, and/or defibrillator, that delivers, or provides, electrical signals (e.g., paces, etc.) to and/or senses electrical signals from the heart 12 of the patient 14 via electrodes coupled to one or more of the leads 18, 20, 22. The IMD 16 is not, however, limited to a particular type of cardiac pacing or defibrillating device and may include one or more lead or leadless devices and/or can be configured to provide conduction system pacing therapy.

The leads 18, 20, 22 extend into the heart 12 of the patient 14 to sense electrical activity of the heart 12 and/or to deliver electrical stimulation to the heart 12. In the example shown in FIG. 12, the right ventricular (RV) lead 18 extends through one or more veins (not shown), the superior vena cava (not shown), and the right atrium 26, and into the right ventricle 28. The left ventricular (LV) coronary sinus lead 20 extends through one or more veins, the vena cava, the right atrium 26, and into the coronary sinus 30 to a region adjacent to the free wall of the left ventricle 32 of the heart 12. The right atrial (RA) lead 22 extends through one or more veins and the vena cava, and into the right atrium 26 of the heart 12.

The IMD 16 may sense, among other things, electrical signals attendant to the depolarization and repolarization of the heart 12 via electrodes coupled to at least one of the leads 18, 20, 22. In one or more embodiments, the IMD 16 provides pacing therapy (e.g., pacing pulses) to the heart 12 based on the electrical signals sensed within the heart 12. The IMD 16 may be operable to adjust one or more parameters associated with the pacing therapy such as, e.g., A-V delay and other various timings, pulse wide, amplitude, voltage, burst length, etc. Further, the IMD 16 may be operable to use various electrode configurations to deliver pacing therapy, which may be unipolar, bipolar, quadripolar, or further multipolar. For example, a multipolar lead may include several electrodes that can be used for delivering pacing therapy. Hence, a multipolar lead system may provide, or offer, multiple electrical vectors to pace from. A pacing vector may include at least one cathode, which may be at least one electrode located on at least one lead, and at least one anode, which may be at least one electrode located on at least one lead (e.g., the same lead, or a different lead) and/or on the casing, or can, of the IMD. While improvement in cardiac function as a result of the pacing therapy may primarily depend on the cathode, the electrical parameters like impedance, pacing threshold voltage, current drain, longevity, etc. may be more dependent on the pacing vector, which includes both the cathode and the anode. The IMD 16 may also provide defibrillation therapy and/or cardioversion therapy via electrodes located on at least one of the leads 18, 20, 22. Further, the IMD 16 may detect arrhythmia of the heart 12, such as fibrillation of the ventricles 28, 32, and deliver defibrillation therapy to the heart 12 in the form of electrical pulses. In some examples, IMD 16 may be programmed to deliver a progression of therapies, e.g., pulses with increasing energy levels, until a fibrillation of heart 12 is stopped.

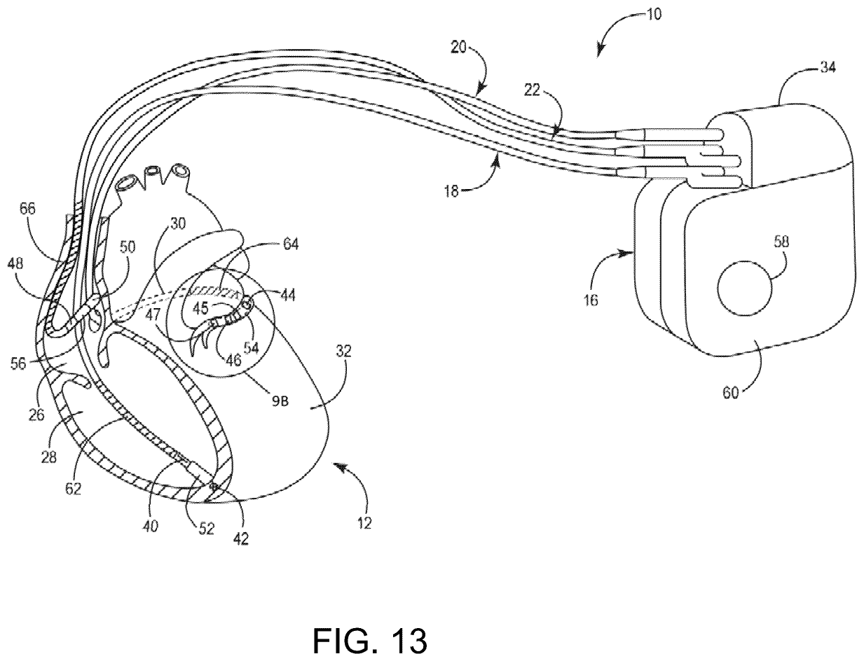

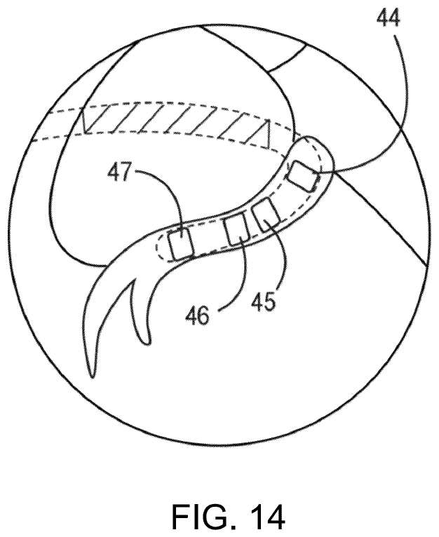

FIGS. 13-14 are conceptual diagrams illustrating the IMD 16 and the leads 18, 20, 22 of therapy system 8 of FIG. 12 in more detail. The leads 18, 20, 22 may be electrically coupled to a therapy delivery module (e.g., for delivery of pacing therapy), a sensing module (e.g., for sensing one or more signals from one or more electrodes), and/or any other modules of the IMD 16 via a connector block 34. In some examples, the proximal ends of the leads 18, 20, 22 may include electrical contacts that electrically couple to respective electrical contacts within the connector block 34 of the IMD 16. In addition, in some examples, the leads 18, 20, 22 may be mechanically coupled to the connector block 34 with the aid of set screws, connection pins, or another suitable mechanical coupling mechanism.

Each of the leads 18, 20, 22 includes an elongated insulative lead body, which may carry conductors (e.g., concentric coiled conductors, straight conductors, etc.) separated from one another by insulation (e.g., tubular insulative sheaths). In the illustrated example, bipolar electrodes 40, 42 are located proximate to a distal end of the lead 18. In addition, bipolar electrodes 44, 45, 46, 47 are located proximate to a distal end of the lead 20 and bipolar electrodes 48, 50 are located proximate to a distal end of the lead 22.

The electrodes 40, 44, 45, 46, 47, 48 may take the form of ring electrodes, and the electrodes 42, 50 may take the form of extendable helix tip electrodes mounted retractably within the insulative electrode heads 52, 54, 56, respectively. Each of the electrodes 40, 42, 44, 45, 46, 47, 48, 50 may be electrically coupled to a respective one of the conductors (e.g., coiled and/or straight) within the lead body of its associated lead 18, 20, 22, and thereby coupled to a respective one of the electrical contacts on the proximal end of the leads 18, 20, 22.

Additionally, electrodes 44, 45, 46 and 47 may have an electrode surface area of about 5.3 mm2 to about 5.8 mm². Electrodes 44, 45, 46, and 47 may also be referred to as LV1, LV2, LV3, and LV4, respectively. The LV electrodes (i.e., left ventricle electrode 1 (LV1) 44, left ventricle electrode 2 (LV2) 45, left ventricle electrode 3 (LV3) 46, and left ventricle 4 (LV4) 47, etc.) on the lead 20 can be spaced apart at variable distances. For example, electrode 44 may be a distance of, e.g., about 21 millimeters (mm), away from electrode 45, electrodes 45 and 46 may be spaced a distance of, e.g., about 1.3 mm to about 1.5 mm, away from each other, and electrodes 46 and 47 may be spaced a distance of, e.g. 20 mm to about 21 mm, away from each other.

The electrodes 40, 42, 44, 45, 46, 47, 48, 50 may further be used to sense electrical signals (e.g., morphological waveforms within electrograms (EGM)) attendant to the depolarization and repolarization of the heart 12. The electrical signals are conducted to the IMD 16 via the respective leads 18, 20, 22. In some examples, the IMD 16 may also deliver pacing pulses via the electrodes 40, 42, 44, 45, 46, 47, 48, 50 to cause depolarization of cardiac tissue of the patient's heart 12. In one or more embodiments, as illustrated in FIG. 13, the IMD 16 includes one or more housing electrodes, such as housing electrode 58, which may be formed integrally with an outer surface of a housing 60 (e.g., hermetically-sealed housing) of the IMD 16 or otherwise coupled to the housing 60. Any of the electrodes 40, 42, 44, 45, 46, 47, 48, 50 may be used for unipolar sensing or pacing in combination with the housing electrode 58. It is generally understood by those skilled in the art that other electrodes can also be selected to define, or be used for, pacing and sensing vectors. Further, any of electrodes 40, 42, 44, 45, 46, 47, 48, 50, 58, when not being used to deliver pacing therapy, may be used to sense electrical activity during pacing therapy.

As described in further detail with reference to FIG. 13, the housing 60 may enclose a therapy delivery module that may include a stimulation generator for generating cardiac pacing pulses and defibrillation or cardioversion shocks, as well as a sensing module for monitoring the electrical signals of the patient's heart (e.g., the patient's heart rhythm). The leads 18, 20, 22 may also include elongated electrodes 62, 64, 66, respectively, which may take the form of a coil. The IMD 16 may deliver defibrillation shocks to the heart 12 via any combination of the elongated electrodes 62, 64, 66 and the housing electrode 58. The electrodes 58, 62, 64, 66 may also be used to deliver cardioversion pulses to the heart 12. Further, the electrodes 62, 64, 66 may be fabricated from any suitable electrically conductive material, such as, but not limited to, platinum, platinum alloy, and/or other materials known to be usable in implantable defibrillation electrodes. Since electrodes 62, 64, 66 are not generally configured to deliver pacing therapy, any of electrodes 62, 64, 66 may be used to sense electrical activity and may be used in combination with any of electrodes 40, 42, 44, 45, 46, 47, 48, 50, 58. In at least one embodiment, the RV elongated electrode 62 may be used to sense electrical activity of a patient's heart during the delivery of pacing therapy (e.g., in combination with the housing electrode 58, or defibrillation electrode-to-housing electrode vector).

The configuration of the illustrative therapy system 10 illustrated in FIGS. 12-16 is merely one example. In other examples, the therapy system may include epicardial leads and/or patch electrodes instead of or in addition to the transvenous leads 18, 20, 22 illustrated in FIG. 12. Additionally, in other examples, the therapy system 10 may be implanted in/around the cardiac space without transvenous leads (e.g., leadless/wireless pacing systems) or with leads implanted (e.g., implanted transvenously or using approaches) into the left chambers of the heart (in addition to or replacing the transvenous leads placed into the right chambers of the heart as illustrated in FIG. 12). Further, in one or more embodiments, the IMD 16 need not be implanted within the patient 14. For example, the IMD 16 may deliver various cardiac therapies to the heart 12 via percutaneous leads that extend through the skin of the patient 14 to a variety of positions within or outside of the heart 12. In one or more embodiments, the system 10 may utilize wireless pacing (e.g., using energy transmission to the intracardiac pacing component(s) via ultrasound, inductive coupling, RF, etc.) and sensing cardiac activation using electrodes on the can/housing and/or on subcutaneous leads.

In other examples of therapy systems that provide electrical stimulation therapy to the heart 12, such therapy systems may include any suitable number of leads coupled to the IMD 16, and each of the leads may extend to any location within or proximate to the heart 12. For example, other examples of therapy systems may include three transvenous leads located as illustrated in FIGS. 12-14. Still further, other therapy systems may include a single lead that extends from the IMD 16 into the right atrium 26 or the right ventricle 28, or two leads that extend into a respective one of the right atrium 26 and the right ventricle 28.

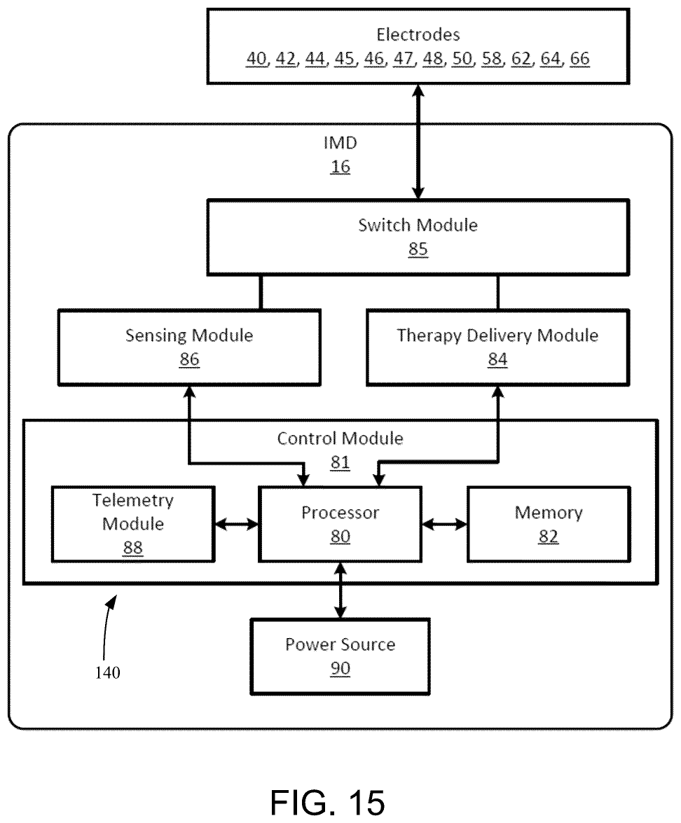

FIG. 15 is a functional block diagram of one illustrative configuration of the IMD 16. As shown, the IMD 16 may include a computing apparatus 140 including a control module 81, a therapy delivery module 84 (e.g., which may include a stimulation generator), a sensing module 86, and a power source 90. In one or more embodiments, the IMD 16 can include an implantable cardiac monitor. In one or more embodiments, two or more IMDs 16 may be utilized with a patient, e.g., an implantable pacemaker and an implantable cardiac monitor. Such two or more IMDs can be in electrical communication with each other and/or with an external device using any suitable wired or wireless technique. In one or more embodiments, a first IMD such as an implantable pacemaker can provide therapeutic electrical stimulation to the heart and a second IMD such as an implantable cardiac monitor can monitor electrical activity of the heart and include a computing apparatus such as computing apparatus 140 that can be configured to utilize the techniques described herein to account for pacing artifacts in a cardiac signal.

The computing apparatus 140 can include any suitable computing apparatus described herein. The computing apparatus 140 can be operatively coupleable to the sensing apparatus (e.g., sensing module 86) and configured to monitor cardiac electrical activity of the patient. The computing apparatus 140 can further be configured to utilize the techniques described herein (e.g., in reference to FIG. 4) to account for pacing artifacts in a cardiac signal.

The computing apparatus 140 includes the control module, or apparatus, 81 which may include a processor 80, memory 82, and a telemetry module, or apparatus, 88. The memory 82 may include computer-readable instructions that, when executed, e.g., by the processor 80, cause the IMD 16 and/or the control module 81 to perform various functions attributed to the IMD 16 and/or the control module 81 described herein. Further, the memory 82 may include any volatile, non-volatile, magnetic, optical, and/or electrical media, such as a random-access memory (RAM), read-only memory (ROM), non-volatile RAM (NVRAM), electrically-erasable programmable ROM (EEPROM), flash memory, and/or any other digital media. An illustrative capture management module may be the left ventricular capture management (LVCM) module described in U.S. Pat. No. 7,684,863 entitled LV THRESHOLD MEASUREMENT AND CAPTURE MANAGEMENT and issued Mar. 23, 2010.

The processor 80 of the control module 81 may include any one or more of a microprocessor, a controller, a digital signal processor (DSP), an application specific integrated circuit (ASIC), a field-programmable gate array (FPGA), and/or equivalent discrete or integrated logic circuitry. In some examples, the processor 80 may include multiple components, such as any combination of one or more microprocessors, one or more controllers, one or more DSPs, one or more ASICs, and/or one or more FPGAs, as well as other discrete or integrated logic circuitry. The functions attributed to the processor 80 herein may be embodied as software, firmware, hardware, or any combination thereof.

The control module 81 may control the therapy delivery module 84 to deliver therapy (e.g., electrical stimulation therapy such as pacing) to the heart 12 according to a selected one or more therapy programs, which may be stored in the memory 82. More, specifically, the control module 81 (e.g., the processor 80) may control various parameters of the electrical stimulus delivered by the therapy delivery module 84 such as, e.g., A-V delays, V-V delays, pacing pulses with the amplitudes, pulse widths, frequency, or electrode polarities, etc., which may be specified by one or more selected therapy programs (e.g., A-V and/or V-V delay adjustment programs, pacing therapy programs, pacing recovery programs, capture management programs, etc.). As shown, the therapy delivery module 84 is electrically coupled to electrodes 40, 42, 44, 45, 46, 47, 48, 50, 58, 62, 64, 66, e.g., via conductors of the respective lead 18, 20, 22, or, in the case of housing electrode 58, via an electrical conductor disposed within housing 60 of IMD 16. Therapy delivery module 84 may be configured to generate and deliver electrical stimulation therapy such as pacing therapy to the heart 12 using one or more of the electrodes 40, 42, 44, 45, 46, 47, 48, 50, 58, 62, 64, 66.

For example, therapy delivery module 84 may deliver pacing stimulus (e.g., pacing pulses) via ring electrodes 40, 44, 45, 46, 47, 48 coupled to leads 18, 20, 22 and/or helical tip electrodes 42, 50 of leads 18, 22. Further, for example, therapy delivery module 84 may deliver defibrillation shocks to heart 12 via at least two of electrodes 58, 62, 64, 66. In some examples, therapy delivery module 84 may be configured to deliver pacing, cardioversion, or defibrillation stimulation in the form of electrical pulses. In other examples, therapy delivery module 84 may be configured deliver one or more of these types of stimulation in the form of other signals, such as sine waves, square waves, and/or other substantially continuous time signals.

The IMD 16 may further include a switch module 85 and the control module 81 (e.g., the processor 80) may use the switch module 85 to select, e.g., via a data/address bus, which of the available electrodes are used to deliver therapy such as pacing pulses for pacing therapy, or which of the available electrodes are used for sensing. The switch module 85 may include a switch array, switch matrix, multiplexer, or any other type of switching device suitable to selectively couple the sensing module 86 and/or the therapy delivery module 84 to one or more selected electrodes. More specifically, the therapy delivery module 84 may include a plurality of pacing output circuits. Each pacing output circuit of the plurality of pacing output circuits may be selectively coupled, e.g., using the switch module 85, to one or more of the electrodes 40, 42, 44, 45, 46, 47, 48, 50, 58, 62, 64, 66 (e.g., a pair of electrodes for delivery of therapy to a bipolar or multipolar pacing vector). In other words, each electrode can be selectively coupled to one of the pacing output circuits of the therapy delivery module using the switching module 85.

The sensing module 86 is coupled (e.g., electrically coupled) to sensing apparatus, which may include, among additional sensing apparatus, the electrodes 40, 42, 44, 45, 46, 47, 48, 50, 58, 62, 64, 66 to monitor cardiac electrical activity of the heart 12, e.g., electrocardiogram (ECG)/electrogram (EGM) signals, etc. The ECG/EGM signals may be used to measure or monitor activation times (e.g., ventricular activations times, etc.), heart rate (HR), heart rate variability (HRV), heart rate turbulence (HRT), deceleration/acceleration capacity, deceleration sequence incidence, T-wave altemans (TWA), P-wave to P-wave intervals (also referred to as the P-P intervals or A-A intervals), R-wave to R-wave intervals (also referred to as the R-R intervals or V-V intervals), P-wave to QRS complex intervals (also referred to as the P-R intervals, A-V intervals, or P-Q intervals), QRS-complex morphology, ST segment (i.e., the segment that connects the QRS complex and the T-wave), T-wave changes, QT intervals, electrical vectors, etc.

The switch module 85 may also be used with the sensing module 86 to select which of the available electrodes are used, or enabled, to, e.g., sense electrical activity of the patient's heart (e.g., one or more electrical vectors of the patient's heart using any combination of the electrodes 40, 42, 44, 45, 46, 47, 48, 50, 58, 62, 64, 66). Likewise, the switch module 85 may also be used with the sensing module 86 to select which of the available electrodes are not to be used (e.g., disabled) to, e.g., sense electrical activity of the patient's heart (e.g., one or more electrical vectors of the patient's heart using any combination of the electrodes 40, 42, 44, 45, 46, 47, 48, 50, 58, 62, 64, 66), etc. In one or more embodiments, the control module 81 may select the electrodes that function as sensing electrodes via the switch module within the sensing module 86, e.g., by providing signals via a data/address bus.

In one or more embodiments, sensing module 86 includes a channel that includes an amplifier with a relatively wider pass band than the R-wave or P-wave amplifiers. Signals from the selected sensing electrodes may be provided to a multiplexer, and thereafter converted to multi-bit digital signals by an analog-to-digital converter for storage in memory 82, e.g., as an electrogram (EGM). In one or more embodiments, the storage of such EGMs in memory 82 may be under the control of a direct memory access circuit.

In one or more embodiments, the control module 81 may operate as an interrupt-driven device and may be responsive to interrupts from pacer timing and control module, where the interrupts may correspond to the occurrences of sensed P-waves and R-waves and the generation of cardiac pacing pulses. Any necessary mathematical calculations may be performed by the processor 80 and any updating of the values or intervals controlled by the pacer timing and control module may take place following such interrupts. A portion of memory 82 may be configured as a plurality of recirculating buffers, capable of holding one or more series of measured intervals, which may be analyzed by, e.g., the processor 80 in response to the occurrence of a pace or sense interrupt to determine whether the patient's heart 12 is presently exhibiting atrial or ventricular tachyarrhythmia.

The telemetry module 88 of the control module 81 may include any suitable hardware, firmware, software, or any combination thereof for communicating with another device, such as a programmer. For example, under the control of the processor 80, the telemetry module 88 may receive downlink telemetry from and send uplink telemetry to a programmer with the aid of an antenna, which may be internal and/or external. The processor 80 may provide the data to be uplinked to a programmer and the control signals for the telemetry circuit within the telemetry module 88, e.g., via an address/data bus. In some examples, the telemetry module 88 may provide received data to the processor 80 via a multiplexer.

The various components of the IMD 16 are further coupled to a power source 90, which may include a rechargeable or non-rechargeable battery. A non-rechargeable battery may be selected to last for several years, while a rechargeable battery may be inductively charged from an external device, e.g., on a daily or weekly basis.

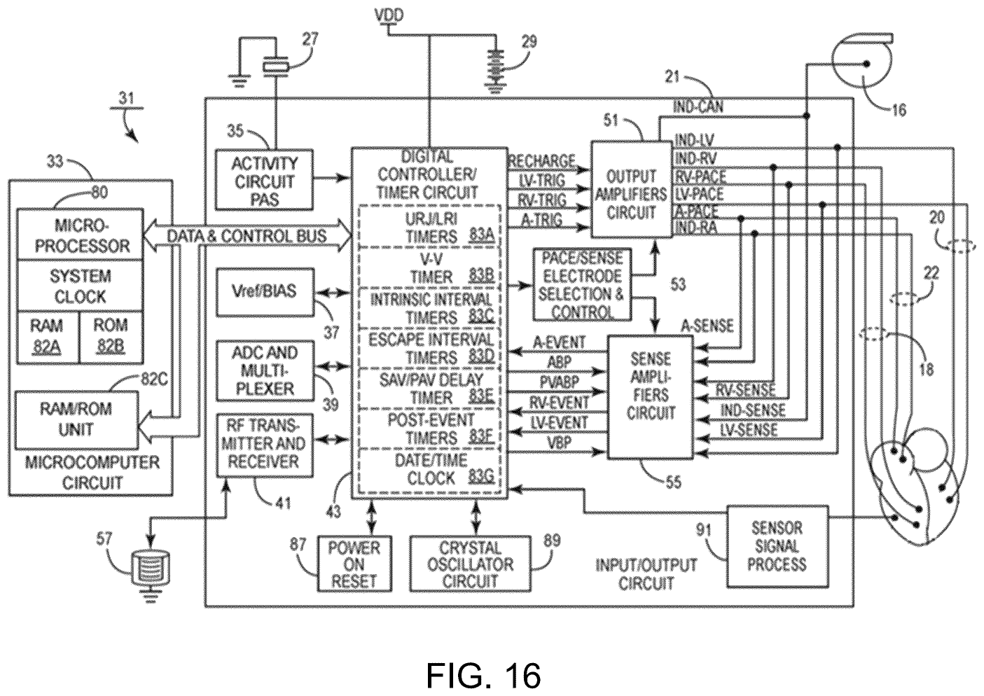

FIG. 16 is another embodiment of a functional block diagram for IMD 16 that depicts bipolar RA lead 22, bipolar RV lead 18, and bipolar LV CS lead 20 without the LA CS pace/sense electrodes and coupled with an implantable pulse generator (IPG) circuit 31 having programmable modes and parameters of a bi-ventricular DDD/R type known in the pacing art. In turn, the sensor signal processing circuit 91 indirectly couples to the timing circuit 43 and via data and control bus to microcomputer circuitry 33. The IPG circuit 31 is illustrated in a functional block diagram divided generally into a microcomputer circuit 33 and a pacing circuit 21. The pacing circuit 21 includes the digital controller/timer circuit 43, the output amplifiers circuit 51, the sense amplifiers circuit 55, the RF telemetry transceiver 41, the activity sensor circuit 35 as well as a number of other circuits and components described below.

Crystal oscillator circuit 89 provides the basic timing clock for the pacing circuit 21 while battery 29 provides power. Power-on-reset circuit 87 responds to initial connection of the circuit to the battery for defining an initial operating condition and similarly, resets the operative state of the device in response to detection of a low battery condition. Reference mode circuit 37 generates stable voltage reference and currents for the analog circuits within the pacing circuit 21. Analog-to-digital converter (ADC) and multiplexer circuit 39 digitize analog signals and voltage to provide, e.g., real time telemetry of cardiac signals from sense amplifiers 55 for uplink transmission via RF transmitter and receiver circuit 41. Voltage reference and bias circuit 37, ADC and multiplexer 39, power-on-reset circuit 87, and crystal oscillator circuit 89 may correspond to any of those used in illustrative implantable cardiac pacemakers.

If the IPG is programmed to a rate responsive mode, the signals output by one or more physiologic sensors are employed as a rate control parameter (RCP) to derive a physiologic escape interval. For example, the escape interval is adjusted proportionally to the patient's activity level developed in the patient activity sensor (PAS) circuit 35 in the depicted, illustrative IPG circuit 31. The patient activity sensor 27 is coupled to the IPG housing and may take the form of a piezoelectric crystal transducer. The output signal of the patient activity sensor 27 may be processed and used as an RCP. Sensor 27 generates electrical signals in response to sensed physical activity that are processed by activity circuit 35 and provided to digital controller/timer circuit 43. Activity circuit 35 and associated sensor 27 may correspond to the circuitry disclosed in U.S. Pat. No. 5,052,388 entitled METHOD AND APPARATUS FOR IMPLEMENTING ACTIVITY SENSING IN A PULSE GENERATOR and issued on Oct. 1, 1991, and U.S. Pat. No. 4,428,378 entitled RATE ADAPTIVE PACER and issued on Jan. 31, 1984. Similarly, the illustrative systems, apparatus, and methods described herein may be practiced in conjunction with alternate types of sensors such as oxygenation sensors, pressure sensors, pH sensors, and respiration sensors, for use in providing rate responsive pacing capabilities. Alternately, QT time may be used as a rate indicating parameter, in which case no extra sensor is required. Similarly, the illustrative embodiments described herein may also be practiced in non-rate responsive pacemakers.

Data transmission to and from the external programmer is accomplished by way of the telemetry antenna 57 and an associated RF transceiver 41, which serves both to demodulate received downlink telemetry and to transmit uplink telemetry. Uplink telemetry capabilities may include the ability to transmit stored digital information, e.g., operating modes and parameters, EGM histograms, and other events, as well as real time EGMs of atrial and/or ventricular electrical activity and marker channel pulses indicating the occurrence of sensed and paced depolarizations in the atrium and ventricle.

Microcomputer 33 contains a microprocessor 80 and associated system clock and on-processor RAM and ROM chips 82A and 82B, respectively. In addition, microcomputer circuit 33 includes a separate RAM/ROM chip 82C to provide additional memory capacity. Microprocessor 80 normally operates in a reduced power consumption mode and is interrupt driven. Microprocessor 80 is awakened in response to defined interrupt events, which may include A-TRIG, RV-TRIG, LV-TRIG signals generated by timers in digital timer/controller circuit 43 and A-EVENT, RV-EVENT, and LV-EVENT signals generated by sense amplifiers circuit 55, among others. The specific values of the intervals and delays timed out by digital controller/timer circuit 43 are controlled by the microcomputer circuit 33 by way of data and control bus from programmed-in parameter values and operating modes. In addition, if programmed to operate as a rate responsive pacemaker, a timed interrupt, e.g., every cycle or every two seconds, may be provided to allow the microprocessor to analyze the activity sensor data and update the basic A-A, V-A, or V-V escape interval, as applicable. In addition, the microprocessor 80 may also serve to define variable, operative A-V delay intervals, V-V delay intervals, and the energy delivered to each ventricle and/or atrium.

In one embodiment, microprocessor 80 is a custom microprocessor adapted to fetch and execute instructions stored in RAM/ROM unit 82 in a conventional manner. It is contemplated, however, that other implementations may be suitable to practice the present disclosure. For example, an off-the-shelf, commercially available microprocessor or microcontroller, or custom application-specific, hardwired logic, or state-machine type circuit may perform the functions of microprocessor 80.

Digital controller/timer circuit 43 operates under the general control of the microcomputer 33 to control timing and other functions within the pacing circuit 21 and includes a set of timing and associated logic circuits of which certain ones pertinent to the present disclosure are depicted. The depicted timing circuits include URI/LRI timers 83A, V-V delay timer 83B, intrinsic interval timers 83C for timing elapsed V-EVENT to V-EVENT intervals or V-EVENT to A-EVENT intervals or the V-V conduction interval, escape interval timers 83D for timing A-A, V-A, and/or V-V pacing escape intervals, an A-V delay interval timer 83E for timing the A-LVp delay (or A-RVp delay) from a preceding A-EVENT or A-TRIG, a post-ventricular timer 83F for timing post-ventricular time periods, and a date/time clock 83G.

The A-V delay interval timer 83E is loaded with an appropriate delay interval for one ventricular chamber (e.g., either an A-RVp delay or an A-LVp) to time-out starting from a preceding A-PACE or A-EVENT. The interval timer 83E triggers pacing stimulus delivery and can be based on one or more prior cardiac cycles (or from a data set empirically derived for a given patient).

The post-event timer 83F times out the post-ventricular time period following an RV-EVENT or LV-EVENT or a RV-TRIG or LV-TRIG and post-atrial time periods following an A-EVENT or A-TRIG. The durations of the post-event time periods may also be selected as programmable parameters stored in the microcomputer 33. The post-ventricular time periods include the PVARP, a post-atrial ventricular blanking period (PAVBP), a ventricular blanking period (VBP), a post-ventricular atrial blanking period (PVARP) and a ventricular refractory period (VRP) although other periods can be suitably defined depending, at least in part, on the operative circuitry employed in the pacing engine. The post-atrial time periods include an atrial refractory period (ARP) during which an A-EVENT is ignored for the purpose of resetting any A-V delay, and an atrial blanking period (ABP) during which atrial sensing is disabled. It should be noted that the starting of the post-atrial time periods and the A-V delays can be commenced substantially simultaneously with the start or end of each A-EVENT or A-TRIG or, in the latter case, upon the end of the A-PACE which may follow the A-TRIG. Similarly, the starting of the post-ventricular time periods and the V-A escape interval can be commenced substantially simultaneously with the start or end of the V-EVENT or V-TRIG or, in the latter case, upon the end of the V-PACE which may follow the V-TRIG. The microprocessor 80 also optionally calculates A-V delays, V-V delays, post-ventricular time periods, and post-atrial time periods that vary with the sensor-based escape interval established in response to the RCP(s) and/or with the intrinsic atrial and/or ventricular rate.

The output amplifiers circuit 51 contains a RA pace pulse generator (and a LA pace pulse generator if LA pacing is provided), a RV pace pulse generator, a LV pace pulse generator, and/or any other pulse generator configured to provide atrial and ventricular pacing. To trigger generation of an RV-PACE or LV-PACE pulse, digital controller/timer circuit 43 generates the RV-TRIG signal at the time-out of the A-RVp delay (in the case of RV pre-excitation) or the LV-TRIG at the time-out of the A-LVp delay (in the case of LV pre-excitation) provided by A-V delay interval timer 83E (or the V-V delay timer 83B). Similarly, digital controller/timer circuit 43 generates an RA-TRIG signal that triggers output of an RA-PACE pulse (or an LA-TRIG signal that triggers output of an LA-PACE pulse, if provided) at the end of the V-A escape interval timed by escape interval timers 83D.

The output amplifiers circuit 51 includes switching circuits for coupling selected pace electrode pairs from among the lead conductors and the IND-CAN electrode 20 to the RA pace pulse generator (and LA pace pulse generator if provided), RV pace pulse generator and LV pace pulse generator. Pace/sense electrode pair selection and control circuit 53 selects lead conductors and associated pace electrode pairs to be coupled with the atrial and ventricular output amplifiers within output amplifiers circuit 51 for accomplishing RA, LA, RV and LV pacing.

The sense amplifiers circuit 55 contains sense amplifiers for atrial and ventricular pacing and sensing. High impedance P-wave and R-wave sense amplifiers may be used to amplify a voltage difference signal that is generated across the sense electrode pairs by the passage of cardiac depolarization wavefronts. The high impedance sense amplifiers use high gain to amplify the low amplitude signals and rely on pass band filters, time domain filtering and amplitude threshold comparison to discriminate a P-wave or R-wave from background electrical noise. Digital controller/timer circuit 43 controls sensitivity settings of the atrial and ventricular sense amplifiers 55.

The sense amplifiers may be uncoupled from the sense electrodes during the blanking periods before, during, and after delivery of a pace pulse to any of the pace electrodes of the pacing system to avoid saturation of the sense amplifiers. The sense amplifiers circuit 55 includes blanking circuits for uncoupling the selected pairs of the lead conductors and the IND-CAN electrode 20 from the inputs of the RA sense amplifier (and LA sense amplifier if provided), RV sense amplifier and LV sense amplifier during the ABP, PVABP and VBP. The sense amplifiers circuit 55 also includes switching circuits for coupling selected sense electrode lead conductors and the IND-CAN electrode 20 to the RA sense amplifier (and LA sense amplifier if provided), RV sense amplifier and LV sense amplifier. Again, sense electrode selection and control circuit 53 selects conductors and associated sense electrode pairs to be coupled with the atrial and ventricular sense amplifiers within the output amplifiers circuit 51 and sense amplifiers circuit 55 for accomplishing RA, LA, RV, and LV sensing along desired unipolar and bipolar sensing vectors.

Right atrial depolarizations or P-waves in the RA-SENSE signal that are sensed by the RA sense amplifier result in a RA-EVENT signal that is communicated to the digital controller/timer circuit 43. Similarly, left atrial depolarizations or P-waves in the LA-SENSE signal that are sensed by the LA sense amplifier, if provided, result in a LA-EVENT signal that is communicated to the digital controller/timer circuit 43. Ventricular depolarizations or R-waves in the RV-SENSE signal are sensed by a ventricular sense amplifier result in an RV-EVENT signal that is communicated to the digital controller/timer circuit 43. Similarly, ventricular depolarizations or R-waves in the LV-SENSE signal are sensed by a ventricular sense amplifier result in an LV-EVENT signal that is communicated to the digital controller/timer circuit 43. The RV-EVENT, LV-EVENT, and RA-EVENT, LA-SENSE signals may be refractory or non-refractory and can inadvertently be triggered by electrical noise signals or aberrantly conducted depolarization waves rather than true R-waves or P-waves.

The techniques described in this disclosure, including those attributed to the IMD 16, the computing apparatus 140, and/or various constituent components, may be implemented, at least in part, in hardware, software, firmware, or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices, or other devices. The term “module,” “processor,” or “processing circuitry” may generally refer to any of the foregoing logic circuitry, alone or in combination with other logic circuitry, or any other equivalent circuitry.

Such hardware, software, and/or firmware may be implemented within the same device or within separate devices to support the various operations and functions described in this disclosure. In addition, any of the described units, modules, or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed by processing circuitry and/or one or more processors to support one or more aspects of the functionality described in this disclosure.

The invention is defined in the claims. However, below there is provided a non-exhaustive listing of non-limiting examples. Any one or more of the features of these examples may be combined with any one or more features of another example, embodiment, or aspect described herein.

Example Ex1. A system that includes a sensing apparatus configured to monitor cardiac electrical activity of a patient, and a computing apparatus that includes one or more processors and operatively coupled to the sensing apparatus. The computing apparatus is configured to monitor cardiac electrical activity using the sensing apparatus to generate a cardiac signal over time, detect a pacing artifact in the cardiac signal, and determine to account for the pacing artifact when using the cardiac signal based on at least one pacing artifact characteristic of the pacing artifact in the cardiac signal. The computing apparatus is further configured to account for the pacing artifact when using the cardiac signal if it is determined to account for the pacing artifact. Accounting for the pacing artifact includes removing the pacing artifact from the cardiac signal between a first data point at time T1 and a second data point at time T2 of the cardiac signal, and replacing the removed pacing artifact in the cardiac signal with replacement data points disposed along a straight line that extends between the first data point and the second data point of the cardiac signal to provide an optimized cardiac signal.

Example Ex2. The system of Ex1, where the computing apparatus is further configured to sample the cardiac signal with a wide bandpass filter prior to detecting the pacing artifact.

Example Ex3. The system of Ex2, where the wide bandpass filter includes a bandwidth of at least 400 Hz.

Example Ex4. The system of Ex3, where the bandwidth of the wide bandpass filter is no greater than 2 kHz.

Example Ex5. The system of Ex3, where the bandwidth of the wide bandpass filter is no greater than 1 kHz.

Example Ex6. The system of any one of Ex1 to Ex5, where the at least one pacing artifact characteristic of the pacing artifact includes a dV/dt that is greater than a threshold.

Example Ex7. The system of Ex6, where the threshold is at least 0.1 V/sec and no greater than 1000 V/sec.

Example Ex8. The system of Ex7, where the threshold is at least 1 V/sec and no greater than 100 V/sec.

Example Ex9. The system of any one of Ex1 to Ex5, where the at least one pacing artifact characteristic of the pacing artifact includes a maximum pacing spike amplitude.

Example Ex10. The system of any one of Ex1 to Ex5, where the at least one pacing artifact characteristic of the pacing artifact includes a pacing recharge time.

Example Ex11. The system of any one of Ex1 to Ex10, where the computing apparatus is further configured to mark the pacing artifact in the cardiac signal.

Example Ex12. The system of Ex11, where marking the pacing artifact in the cardiac signal includes indicating at least one of a position, an activation time, a duration, a maximum spike amplitude, or a pacing recharge time of the pacing artifact.

Example Ex13. The system of any one of Ex1 to Ex12, where an interval between T1 and T2 is at least 10 milliseconds.

Example Ex14. The system of Ex13, where the interval between T1 and T2 is no greater than 100 milliseconds.

Example Ex15. The system of Ex13, where the interval between T1 and T2 is no greater than 50 milliseconds.

Example Ex16. The system of Ex13, where the interval between T1 and T2 is no greater than 30 milliseconds.

Example Ex17. The system of any one of Ex1 to Ex16, where the computing apparatus is further configured to sample the optimized cardiac signal with a narrow bandpass filter.

Example Ex18. The system of any one of Ex1 to Ex17, where the sensing apparatus includes an ECG lead.

Example Ex19. The system of any one of Ex1 to Ex18, where the computing apparatus is further configured to alert a user of the pacing artifact.

Example Ex20. An implantable medical device that includes a housing, and a computing apparatus disposed within the housing and including one or more processors, where the computing apparatus is operatively coupleable to a sensing apparatus configured to monitor cardiac electrical activity of a patient. The computing apparatus is further configured to monitor cardiac electrical activity using the sensing apparatus to generate a cardiac signal over time, detect a pacing artifact in the cardiac signal, determine to account for the pacing artifact when using the cardiac signal based on at least one pacing artifact characteristic of the pacing artifact in the cardiac signal, and account for the pacing artifact when using the cardiac signal if it is determined to account for the pacing artifact. Accounting for the pacing artifact includes removing the pacing artifact from the cardiac signal between a first data point at time T1 and a second data point at time T2 of the cardiac signal, and replacing the removed pacing artifact in the cardiac signal with replacement data points disposed along a straight line that extends between the first data point and the second data point of the cardiac signal to provide an optimized cardiac signal.

Example Ex21. The device of Ex20, where the computing apparatus is further configured to sample the cardiac signal with a wide bandpass filter prior to detecting the pacing artifact.

Example Ex22. The device of Ex21, where the wide bandpass filter includes a bandwidth of at least 400 Hz.

Example Ex23. The device of Ex22, where the bandwidth of the wide bandpass filter is no greater than 2 kHz.

Example Ex24. The device of Ex22, where the bandwidth of the wide bandpass filter is no greater than 1 kHz.

Example Ex25. The device of any one of Ex20 to Ex24, where the at least one pacing artifact characteristic of the pacing artifact includes a dV/dt that is greater than a threshold.

Example Ex26. The device of Ex25, where the threshold is at least 0.1 V/sec and no greater than 1000 V/sec.

Example Ex27. The device of Ex26, where the threshold is at least 1 V/sec and no greater than 100 V/sec.

Example Ex28. The device of any one of Ex20 to Ex24, where the at least one pacing artifact characteristic of the pacing artifact includes a maximum pacing spike amplitude.

Example Ex29. The device of any one of Ex20 to Ex24, where the at least one pacing artifact characteristic of the pacing artifact includes a pacing recharge time.

Example Ex30. The device of any one of Ex20 to Ex29, where the computing apparatus is further configured to mark the pacing artifact in the cardiac signal.

Example Ex 31. The device of Ex30, where marking the pacing artifact in the cardiac signal includes indicating at least one of a position, an activation time, a duration, a maximum spike amplitude, or a pacing recharge time of the pacing artifact.

Example Ex32. The device of any one of Ex20 to Ex31, where an interval between T1 and T2 is at least 10 milliseconds.

Example Ex33. The device of Ex32, where the interval between T1 and T2 is no greater than 100 milliseconds.

Example Ex34. The device of Ex32, where the interval between T1 and T2 is no greater than 50 milliseconds.

Example Ex35. The device of Ex32, where the interval between T1 and T2 is no greater than 30 milliseconds.

Example Ex36. The device of any one of Ex20 to Ex35, where the computing apparatus is further configured to sample the optimized cardiac signal with a narrow bandpass filter.

Example Ex37. The device of any one of Ex20 to Ex36, where the device further includes the sensing apparatus, and where the sensing apparatus includes an electrode disposed on or at least partially within the housing.

Example Ex38. The device of any one of Ex20 to Ex37, where the computing apparatus is further configured to alert a user of the pacing artifact.

Example Ex39. A method that includes monitoring cardiac electrical activity using a sensing apparatus to generate a cardiac signal over time, detecting a pacing artifact in the cardiac signal, and determining to account for the pacing artifact when using the cardiac signal based on at least one pacing artifact characteristic of the pacing artifact in the cardiac signal. The method further includes accounting for the pacing artifact when using the cardiac signal if it is determined to account for the pacing artifact. Accounting for the pacing artifact includes removing the pacing artifact from the cardiac signal between a first data point at time T1 and a second data point at time T2 of the cardiac signal, and replacing the removed pacing artifact in the cardiac signal with replacement data points disposed along a straight line that extends between the first data point and the second data point of the cardiac signal to provide an optimized cardiac signal.

Example Ex40. The method of Ex39, further including sampling the cardiac signal with a wide bandpass filter prior to detecting the pacing artifact.

Example Ex41. The method of Ex40, where the wide bandpass filter includes a bandwidth of at least 400 Hz.

Example Ex42. The method of Ex41, where the bandwidth of the wide bandpass filter is no greater than 2 kHz.

Example Ex43. The method of Ex41, where the bandwidth of the wide bandpass filter is no greater than 1 kHz.

Example Ex44. The method of any one of Ex39 to Ex43, where the at least one pacing artifact characteristic of the pacing artifact includes a dV/dt that is greater than a threshold.

Example Ex45. The method of Ex44, where the threshold is at least 0.1 V/sec and no greater than 1000 V/sec.

Example Ex46. The method of Ex45, where the threshold is at least 1 V/sec and no greater than 100 V/sec.

Example Ex47. The method of any one of Ex39 to Ex43, where the at least one pacing artifact characteristic of the pacing artifact includes a maximum pacing spike amplitude.

Example Ex48. The method of any one of Ex39 to Ex43, where the at least one pacing artifact characteristic of the pacing artifact includes a pacing recharge time.

Example Ex49. The method of any one of Ex39 to Ex48, further including marking the pacing artifact in the cardiac signal.

Example Ex50. The method of Ex49, where marking the pacing artifact in the cardiac signal includes indicating at least one of a position, an activation time, a duration, a maximum spike amplitude, or a pacing recharge time of the pacing artifact.

Example Ex51. The method of any one of Ex39 to Ex50, where an interval between T1 and T2 is at least 10 milliseconds.

Example Ex52. The method of Ex51, where the interval between T1 and T2 is no greater than 100 milliseconds.

Example Ex53. The method of Ex51, where the interval between T1 and T2 is no greater than 50 milliseconds.

Example Ex54. The method of Ex51, where the interval between T1 and T2 is no greater than 30 milliseconds.

Example Ex55. The method of any one of Ex39 to Ex54, further including sampling the optimized cardiac signal with a narrow bandpass filter.

Example Ex56. The method of any one of Ex40 to Ex55, where the sensing apparatus includes an ECG lead.

Example Ex57. The method of any one of Ex40 to Ex56, further including alerting a user of the pacing artifact.

Example Ex58. A computing apparatus that includes one or more processors and operatively couplable to a sensing apparatus. The computing apparatus is configured to monitor cardiac electrical activity using the sensing apparatus to generate a cardiac signal over time, detect a pacing artifact in the cardiac signal, and determine to account for the pacing artifact when using the cardiac signal based on at least one pacing artifact characteristic of the pacing artifact in the cardiac signal. The computing apparatus is further configured to account for the pacing artifact when using the cardiac signal if it is determined to account for the pacing artifact. Accounting for the pacing artifact includes removing the pacing artifact from the cardiac signal between a first data point at time T1 and a second data point at time T2 of the cardiac signal, and replacing the removed pacing artifact in the cardiac signal with replacement data points disposed along a straight line that extends between the first data point and the second data point of the cardiac signal to provide an optimized cardiac signal.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

All references and publications cited herein are expressly incorporated herein by reference in their entirety into this disclosure, except to the extent they may directly contradict this disclosure. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below.

What is claimed is:

1. A system comprising:
a sensing apparatus configured to monitor cardiac electrical activity of a patient; and
a computing apparatus comprising one or more processors and operatively coupled to the sensing apparatus, the computing apparatus configured to:
monitor cardiac electrical activity using the sensing apparatus to generate a cardiac signal over time;
detect a pacing artifact in the cardiac signal;
determine to account for the pacing artifact when using the cardiac signal based on at least one pacing artifact characteristic of the pacing artifact in the cardiac signal; and
account for the pacing artifact when using the cardiac signal if it is determined to account for the pacing artifact, wherein accounting for the pacing artifact comprises:
removing the pacing artifact from the cardiac signal between a first data point at time T1 and a second data point at time T2 of the cardiac signal; and
replacing the removed pacing artifact in the cardiac signal with replacement data points disposed along a straight line that extends between the first data point and the second data point of the cardiac signal to provide an optimized cardiac signal.

2. The system of claim 1, wherein the computing apparatus is further configured to sample the cardiac signal with a wide bandpass filter prior to detecting the pacing artifact.

3. The system of claim 2, wherein the wide bandpass filter comprises a bandwidth between about 400 Hz and 2 kHz.

4. The system of claim 1, wherein the at least one pacing artifact characteristic of the pacing artifact comprises a dV/dt that is greater than a threshold between about 0.1 V/sec and about 1000 V/sec.

5. The system of claim 1, wherein the at least one pacing artifact characteristic of the pacing artifact comprises at least one of a maximum pacing spike amplitude or a pacing recharge time.

6. The system of claim 1, wherein the computing apparatus is further configured to mark the pacing artifact in the cardiac signal.

7. The system of claim 6, wherein marking the pacing artifact in the cardiac signal comprises indicating at least one of a position, an activation time, a duration, a maximum spike amplitude, or a pacing recharge time of the pacing artifact.

8. The system of claim 1, wherein an interval between T1 and T2 is between about 10 milliseconds and about 100 milliseconds.

9. The system of claim 1, wherein the computing apparatus is further configured to sample the optimized cardiac signal with a narrow bandpass filter.

10. The system of claim 1, comprising an implantable medical device having a housing and the computing apparatus disposed within the housing and operatively coupleable to the sensing apparatus.

12. The system of claim 1, wherein the sensing apparatus comprises an ECG lead.

13. A method comprising:
monitoring cardiac electrical activity using a sensing apparatus to generate a cardiac signal over time;
detecting a pacing artifact in the cardiac signal;
determining to account for the pacing artifact when using the cardiac signal based on at least one pacing artifact characteristic of the pacing artifact in the cardiac signal; and
accounting for the pacing artifact when using the cardiac signal if it is determined to account for the pacing artifact, wherein accounting for the pacing artifact comprises:
removing the pacing artifact from the cardiac signal between a first data point at time T1 and a second data point at time T2 of the cardiac signal; and
replacing the removed pacing artifact in the cardiac signal with replacement data points disposed along a straight line that extends between the first data point and the second data point of the cardiac signal to provide an optimized cardiac signal.

14. The method of claim 13, wherein the computing apparatus is further configured to sample the cardiac signal with a wide bandpass filter prior to detecting the pacing artifact, wherein the wide bandpass filter comprises a bandwidth between about 400 Hz and 2 kHz.

15. The method of claim 13, wherein the at least one pacing artifact characteristic of the pacing artifact comprises a dV/dt that is greater than a threshold between about 0.1 V/sec and about 1000 V/sec.

16. The method of claim 13, wherein the at least one pacing artifact characteristic of the pacing artifact comprises at least one of a maximum pacing spike amplitude or a pacing recharge time.

17. The method of claim 13, further comprising, via the computing apparatus, marking the pacing artifact in the cardiac signal.

18. The method of claim 17, wherein marking the pacing artifact in the cardiac signal comprises indicating at least one of a position, an activation time, a duration, a maximum spike amplitude, or a pacing recharge time of the pacing artifact.

19. The method of claim 13, wherein an interval between T1 and T2 is between about 10 milliseconds and about 100 milliseconds.

20. The method of claim 13, further comprising, via the computing apparatus, sampling the optimized cardiac signal with a narrow bandpass filter.
